(19) United States
(12) Patent Application Publication
(10) Pub. No.: US 2007/0049627 A1
(43) Pub. Date: Mar. 1, 2007

(54) Treating vulvodynia using prodrugs of GABA analogs

(76) Inventor: Pierre V. Tran, San Jose, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/507,659

(22) Filed: Aug. 22, 2006

Related U.S. Application Data
(60) Provisional application No. 60/710,963, filed on Aug. 23, 2005.

(51) Int. Cl. A61K 31/325; A61K 31/44; A61K 31/40

(52) U.S. Cl. 514/357; 514/408; 514/483

(57) ABSTRACT

Methods of using prodrugs of GABA analogs and pharmaceutical compositions thereof to treat vulvodynia in a patient, and pharmaceutical compositions of prodrugs of GABA analogs useful in treating vulvodynia are disclosed.

This application claims the benefit of U.S. Provisional Application No. 60/710,963 filed Aug. 23, 2005, which is incorporated by reference herein in its entirety.

### 1. FIELD

The methods and compositions disclosed herein relate generally to methods of treating vulvodynia in a patient. More specifically, disclosed herein are methods of using prodrugs of GABA analogs and pharmaceutical compositions thereof to treat vulvodynia in patients and pharmaceutical compositions of prodrugs of GABA analogs useful in treating vulvodynia.

### 2. BACKGROUND

Vulvodynia is pain localized to the vulva and is a complex gynecological disorder that is often difficult to diagnose and treat (see e.g., Reviews of Gynecological Practice, 2002, Elsevier Press, Vol. 2, Issues 1-2). While the prevalence of vulvodynia is unknown, it is estimated that lifetime cumulative incidence of chronic vulval pain may be 16%, which suggests that 14 million U.S. women could experience vulvodynia during their lifetime (Harlow et al., J. Am. Med. Womens Assoc. 2003, 58, 82-88).

The vulva is the region between a woman's thighs and includes the labia majora, the labia minor, and the vestibule, an oval-shaped area that goes from the back of the vaginal opening to just below the clitoris and includes the vaginal and urethral openings. The two major vulvar pain conditions are localized vulvodynia and generalized vulvodynia, also referred to as vulvar vestibulitis and dysesthetic vulvodynia, respectively.

The most common type of vulvodynia is vulvar vestibulitis. Women with vulvar vestibulitis experience pain involving and limited to the vestibule and only during or after touch or pressure is applied. Vulvar vestibulitis is characterized by pain, tenderness, vestibular erythema, itching, swelling, excoriation, and/or the exclusion of other causes for vestibular erythema and tenderness such as candidiasis (yeast infections) or herpes infections. The pain in vulvar vestibulitis may be described as sharp, burning, or a sensation of rawness. Women with localized vulvodynia can experience pain during, for example, intercourse, tampon use, wearing binding clothes, and physical activity. Generalized vulvodynia is diffuse pain and/or burning sensation on or around the vulva, the labia majora, labia minor, and/or the vestibule. Some women also experience pain in the clitoris, mons pubis, perineum, and/or the inner thighs. The pain can be constant or intermittent and the symptoms, although not necessarily caused by touch or pressure to the vulva, can be exacerbated by physical contact to the area. As a chronic pain condition, vulvodynia can have a significant impact on a woman's quality of life, often affecting her ability to engage in sexual activity and interfere with daily functioning. These limitations can negatively affect self-image and lead to depression.

Although the causes of vulvodynia are unknown, potential conditions believed to cause vulvodynia include injury to or irritation of the nerves that innervate the vulva, allergic reactions or an abnormal response to environmental irritants, genetic factors associated with susceptibility to chronic vestibular inflammation, high levels of oxalate crystals in the urine, and/or spasms of the muscles that support the pelvic organs. Recent studies suggest that the etiology of vulvodynia is associated with neuropathic processes (Shafik, Eur. J. Obstet. Gynecol Reprod Biol. 1998, 80, 215-20, Cox, Lancet 1995, 345, 53; Ben-David et al., Anesth. Analg. 1999, 89, 1459-60). The role of neuropathic pain in vulvodynia is supported by good response rates when patients are treated for neuropathic pain (Edwards, Am. J. Obstet. Gynecol. 2003, 189, S24-30).

Medications used to treat neuropathic pain have been used to successfully treat vulvodynia (see Haefner et al., J. Lower Genital Tract Disease, 2005, 9(1), 40-51). Oral tricyclic antidepressants such as amitriptyline (Elvalil®, AstraZeneca), nortriptyline (Pamelor®, Novartis), desipramine (Norpramin®, Aventis Pharmaceuticals), and selective serotonin reuptake inhibitors such as venlafaxine (Effexor® XR, Wyeth) have been used to treat vulvar pain with variable success (Murphy et al., Clin Evid. 2002, 1875-77; Stolar et al., J. Obstet Gynaecol. 2002, 159, 316-17; Munday, J. Obstet. Gynaecol. 2001, 21, 610-13; McKay, J. Reprod. Med. 1993, 38, 9-13). Anticonvulsants such as gabapentin (Neurontin®, Pfizer) and carbamazepine (Tegretol®, Novartis) have also been used to treat vulvar pain (Scheinfeld, Intl. J. Dermatology, 2003, 42, 491-95; Bates et al., Intl. J. STD AIDS 2002, 13, 210-212; Ben-David et al., Anesth. Analg. 1999, 89, 1459-1460; Sasaki et al., Tech. Urol. 2001, 7, 47-49).

The γ-aminobutyric acid (γ-aminobutyric acid is abbreviated herein as GABA) analog, gabapentin (1), has been approved in the United States for the treatment of epileptic seizures, diabetic neuropathy, post-herpetic neuralgia, and restless legs syndrome. (Backonja et al., JAMA 1998, 280, 1831-36; Rose et al., Anaesthesia 2002, 57, 451-62). The drug has also shown efficacy in controlled studies for treating neuropathic pain of varying etiologies. Gabapentin has been used to treat a number of other medical disorders (Magnus, Epilepsia 1999, 40, S66-72), including vulvodynia (Ben-David et al., Anesth Analg 1999, 89, 1459-1460; Bates et al., Int. J. STD AIDS 2002, 13, 210-212; and Scheinfeld, Intl J. Dermatology, 2003, 42, 491-495). In one study, patients suffering from chronic vulvar pain and who failed to respond favorably to treatment with the tricyclic antidepressant, amitriptyline, were administered increasing amounts of gabapentin over a 12-week period (Ben-David et al., Anesth Analg. 1999, 89, 1459-1460). Eighty-two percent of the patients experienced partial or complete relief of vulvar pain symptoms. While gabapentin is generally tolerated better and is less sedating than tricyclic antidepressant medications, side effects such as headaches, nausea, vomiting, fatigue, and dizziness were reported in over half the patients.

The broad pharmaceutical activities of GABA analogs such as gabapentin (1) and pregabalin (2): 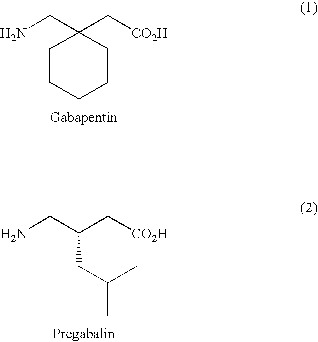
have stimulated intensive interest in preparing related compounds that have superior pharmaceutical properties in comparison to GABA, e.g., the ability to cross the blood brain barrier (see e.g., Satzinger et al., U.S. Pat. No. 4,024,175; Silverman et al., U.S. Pat. No. 5,563,175; Horwell et al., U.S. Pat. No. 6,020,370; Silverman et al., U.S. Pat. No. 6,028,214; Horwell et al., U.S. Pat. No. 6,103,932; Silverman et al., U.S. Pat. No. 6,117,906; Silverman, International Publication No. WO 92/09560; Silverman et al., International Publication No. WO 93/23383; Horwell et al., International Publication No. WO 97/29101, Horwell et al., International Publication No. WO 97/33858; Horwell et al., International Publication No. WO 97/33859; Bryans et al., International Publication No. WO 98/17627; Guglietta et al., International Publication No. WO 99/08671; Bryans et al., International Publication No. WO 99/21824; Bryans et al., International Publication No. WO 99/31057; Belliotti et al., International Publication No. WO 99/31074; Bryans et al., International Publication No. WO 99/31075; Bryans et al., International Publication No. WO 99/61424; Bryans et al., International Publication No. WO 00/15611; Belliot et al., International Publication No. WO 00/31020; Bryans et al., International Publication No. WO 00/50027; and Bryans et al., International Publication No. WO 02/00209).

One significant problem associated with the clinical use of many GABA analogs, including gabapentin and pregabalin, is rapid systemic clearance. Consequently, these drugs require frequent dosing to maintain a therapeutic or prophylactic concentration in the systemic circulation (Bryans et al., Med. Res. Rev. 1999, 19, 149-177). For example, dosing regimens of 300-600 mg doses of gabapentin administered three times per day are typically used for anticonvulsive therapy. Higher doses (1800-3600 mg/day in three or four divided doses) are typically used for the treatment of neuropathic pain states. Doses of gabapentin up to 1200 mg/day with 300 mg administered four times a day have been shown to be effective in treating vulvodynia (Ben-David et al., Anesth, Analg. 1999, 89, 1459-60).

Although oral sustained released formulations are conventionally used to reduce the dosing frequency of drugs that exhibit rapid systemic clearance, oral sustained release formulations of gabapentin and pregabalin have not been developed because these drugs are not absorbed via the large intestine. Rather, these compounds are typically absorbed in the small intestine by one or more amino acid transporters (e.g., the “large neutral amino acid transporter,” see Jezyk et al., Pharm. Res. 1999, 16, 519-526). The limited residence time of both conventional and sustained release oral dosage forms in the proximal absorptive region of the gastrointestinal tract necessitates frequent daily dosing of conventional oral dosage forms of these drugs, and has prevented the successful application of sustained release technologies to many GABA analogs.

One method for overcoming rapid systemic clearance of GABA analogs is to administer an extended release dosage formulation containing a colonically absorbed GABA analog prodrug (Gallop et al., International Publication Nos. WO 02/100347 and WO 02/100349). Sustained release formulations enable the prodrugs to be absorbed over a wider region of the gastrointestinal tract than the parent drug including across the wall of the colon where sustained release oral dosage forms typically spend a significant portion of gastrointestinal transit time. These prodrugs are typically converted to the parent GABA analog upon absorption in vivo.

### 3. SUMMARY

Currently, there is no drug approved by the Food and Drug Administration for treating vulvodynia. Furthermore, therapeutic agents for treating vulvodynia either have significant side effects or are rapidly systemically cleared. Therefore, there is a need in the art for a method of treating vulvodynia by delivering an agent, such as a prodrug of a GABA analog, for example, in an extended release dosage form, with a reduced rate of systemic clearance, and without significant side effects.

Certain embodiments of the present disclosure provide methods of treating vulvodynia in a patient in need of such treatment comprising administering to the patient a therapeutically effective amount of at least one compound chosen from Formula (I) and Formula (II): 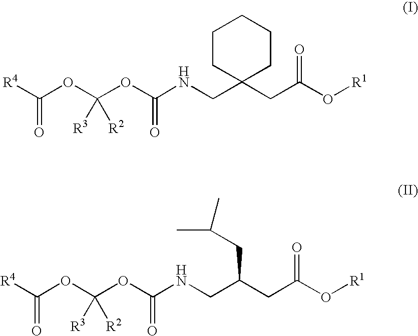
pharmaceutically acceptable salts thereof, pharmaceutically acceptable solvates of any of the foregoing, and pharmaceutically acceptable N-oxides of any of the foregoing, wherein:
R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are each independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and
R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

Certain embodiments of the present disclosure provide methods of treating vulvodynia in a patient in need of such treatment comprising administering to the patient a pharmaceutical composition comprising a therapeutically effective amount of at least one compound chosen from Formula (I) and Formula (II), a pharmaceutically acceptable salts thereof, pharmaceutically acceptable solvates of any of the foregoing, and pharmaceutically acceptable N-oxides of any of the foregoing, and a pharmaceutically acceptable vehicle.

### 4. DETAILED DESCRIPTION

### 4.1 Definitions

“Compounds” refers to GABA analog prodrugs of Formula (I) and Formula (II), wherein compounds of Formula (I) are prodrugs of gabapentin and compounds of Formula (II) are prodrugs of pregabalin. Compounds may be identified either by their chemical structure and/or chemical name. When the chemical structure and chemical name conflict, the chemical structure is determinative of the identity of the compound. The compounds described herein may comprise one or more chiral centers and/or double bonds and therefore, may exist as stereoisomers, such as double-bond isomers (i.e., geometric isomers), enantiomers, and diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Compounds may also exist in several tautomeric forms including the enol form, the keto form, and mixtures of any of the foregoing. Accordingly, the chemical structures depicted herein encompass all possible tautomeric forms of the illustrated compounds. Compounds described herein also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. Certain compounds may exist in multiple crystalline or amorphous forms. All physical forms are equivalent for the uses contemplated herein. Further, it should be understood, when partial structures of the compounds are illustrated, that brackets indicate the point of attachment of the partial structure to the rest of the molecule.

“Alkyl” by itself or as part of another substituent refers to a saturated or unsaturated, branched or straight-chain monovalent hydrocarbon radical derived by the removal of one hydrogen atom from a single carbon atom of a parent alkane, alkene or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but- 1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the terms “alkanyl,” “alkenyl,” and “alkynyl” are used. In certain embodiments, an alkyl group comprises from 1 to 20 carbon atoms, in certain embodiments, from 1 to 6 carbon atoms, and in certain embodiments, from 1 to 3 carbon atoms.

“Alkanyl” by itself or as part of another substituent refers to a saturated branched or straight-chain alkyl radical derived by the removal of one hydrogen atom from a single carbon atom of a parent alkane. Examples of alkanyl groups include, but are not limited to, methanyl; ethanyl; propanyls such as propan-1-yl, and propan-2-yl (isopropyl), etc.; butanyls such as butan-1-yl, butan-2-yl (sec-butyl), 2-methyl-propan-1-yl (isobutyl), and 2-methyl-propan-2-yl (t-butyl), etc.; and the like.

“Alkenyl” by itself or as part of another substituent refers to an unsaturated branched or straight-chain alkyl radical having at least one carbon-carbon double bond derived by the removal of one hydrogen atom from a single carbon atom of a parent alkene. The group may be in either the cis or trans conformation about the double bond(s). Examples of alkenyl groups include, but are not limited to, ethenyl; propenyls such as prop-1-en-1-yl, prop-1-en-2-yl, prop-2-en-1-yl (allyl), and prop-2-en-2-yl,; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl, etc.; and the like.

“Alkynyl” by itself or as part of another substituent refers to an unsaturated branched or straight-chain alkyl radical having at least one carbon-carbon triple bond derived by the removal of one hydrogen atom from a single carbon atom of a parent alkyne. Examples of alkynyl groups include, but are not limited to, ethynyl; propynyls such as prop-1-yn-1-yl, and prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is chosen from hydrogen, alkyl, cycloalkyl, cycloheteroalkyl, aryl, arylalkyl, heteroalkyl, heteroaryl, and heteroarylalkyl as defined herein. Examples of acyl groups include, but are not limited to formyl, acetyl, cyclohexylcarbonyl, cyclohexylmethylcarbonyl, benzoyl, benzylcarbonyl, and the like.

“Alkoxy” by itself or as part of another substituent refers to a radical —OR³¹ where R³¹ is chosen from an alkyl and cycloalkyl group as defined herein. Examples of alkoxy groups include, but are not limited to, methoxy, ethoxy, propoxy, butoxy, cyclohexyloxy, and the like.

“Alkoxycarbonyl” by itself or as part of another substituent refers to a radical —C(O)—OR³² where R³² is chosen from an alkyl and cycloalkyl group as defined herein. Examples of alkoxycarbonyl groups include, but are not limited to, methoxycarbonyl, ethoxycarbonyl, propoxycarbonyl, butoxycarbonyl, cyclohexyloxycarbonyl, and the like.

“Aryl” by itself or as part of another substituent refers to a monovalent aromatic hydrocarbon radical derived by the removal of one hydrogen atom from a single carbon atom of a parent aromatic ring system. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group comprises from 6 to 20 carbon atoms, for example, from 6 to 12 carbon atoms.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, and/or arylalkynyl is used. In certain embodiments, an arylalkyl group is C₆₋₃₀ arylalkyl, e.g., the alkanyl, alkenyl, or alkynyl moiety of the arylalkyl group is C₁₋₁₀ and the aryl moiety is C₆₋₂₀. In certain embodiments, an arylalkyl group is C₆₋₂₀ arylalkyl, e.g., the alkanyl, alkenyl, or alkynyl moiety of the arylalkyl group is C₁₋₈ and the aryl moiety is C₆₋₁₂.

“AUC” is the area under the plasma drug concentration-versus-time curve extrapolated from zero time to infinity.

“Carbamoyl” by itself or as part of another substituent refers to the radical —C(O)NR⁴⁰R⁴¹ where R⁴⁰ and R⁴¹ are independently chosen from hydrogen, alkyl, cycloalkyl, and aryl as defined herein.

“Cmax” is the highest drug concentration observed in plasma following an extravascular dose of drug.

“Cycloalkyl” by itself or as part of another substituent refers to a saturated or partially unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, the cycloalkyl group is C₃₋₁₀ cycloalkyl, for example, C₃₋₇ cycloalkyl.

“Cycloheteroalkyl” by itself or as part of another substituent refers to a saturated or partially unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, quinuclidine, and the like.

“GABA analog” refers to gabapentin (1) or pregabalin (2).

“Heteroalkyl,” “heteroalkanyl,” “heteroalkenyl,” and “heteroalkynyl” by themselves or as part of another substituent refer to alkyl, alkanyl, alkenyl, and alkynyl groups, respectively, in which one or more of the carbon atoms (and any associated hydrogen atoms) are independently replaced with the same or different heteroatomic groups. Typical heteroatomic groups include, but are not limited to, —O—, —S—, —O—O—, —S—S—, —O—S—, —NR⁴²R⁴³, ═N—N═, —N═N—, —N═N—NR⁴⁴R⁴⁵, —PR⁴⁶—, —P(O)₂—, —POR⁴⁷—, —O—P(O)₂—, —SO—, —SO₂—, —SnR⁴⁸R⁴⁹—, and the like, where R⁴², R⁴³, R⁴⁴, R⁴⁵, R⁴⁶, R⁴⁷, R⁴⁸, and R⁴⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

“Heteroaryl” by itself or as part of another substituent refers to a monovalent heteroaromatic radical derived by the removal of one hydrogen atom from a single atom of a parent heteroaromatic ring system. Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-20 membered heteroaryl, such as a 5-10 membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, and/or heterorylalkynyl is used. In certain embodiments, the heteroarylalkyl group is a 6-30 membered heteroarylalkyl, e.g., the alkanyl, alkenyl, or alkynyl moiety of the heteroarylalkyl is 1-10 membered and the heteroaryl moiety is a 5-20-membered heteroaryl, such as, 6-20 membered heteroarylalkyl,e.g., the alkanyl, alkenyl, or alkynyl moiety of the heteroarylalkyl is 1-8 membered and the heteroaryl moiety is a 5-12-membered heteroaryl.

“N-oxide” refers to the zwitterionic nitrogen oxide of a tertiary amine base.

“Parent aromatic ring system” refers to an unsaturated cyclic or polycyclic ring system having a conjugated π electron system. Specifically included within the definition of “parent aromatic ring system” are fused ring systems in which one or more of the rings are aromatic and one or more of the rings are saturated, partially unsaturated, or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms that replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated, partially unsaturated, or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Patient” refers to a mammal, for example a human, such as a human female of any age.

“Pharmaceutically acceptable” refers to approved or approvable by a regulatory agency of a federal or a state government or listed in the U.S. Pharmacopoeia or other generally recognized pharmacopoeia for use in animals, for example, in humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

“Pharmaceutically acceptable vehicle” refers to a diluent, adjuvant, excipient or carrier with which a compound of the invention is administered.

“Pharmaceutical composition” refers to at least one therapeutic compound and at least one pharmaceutically acceptable vehicle, with which the compound is administered to a patient. Pharmaceutical compositions of the present disclosure comprise at least one compound of Formula (I) and/or at least one compound of Formula (II), and at least one pharmaceutically acceptable vehicle.

“Prodrug” refers to a derivative of a drug molecule that requires a transformation within the body to release the active drug. Prodrugs are frequently, although not necessarily, pharmacologically inactive until converted to the parent drug. A hydroxyl-containing drug may be converted to, for example, to a sulfonate, ester or carbonate prodrug, which may be hydrolyzed in vivo to provide the hydroxyl compound. An amino containing drug can be converted, for example, to a carbamate, amide, enamine, imine, N-phosphonyl, N-phosphoryl, or N-sulfenyl prodrug, which may be hydrolyzed in vivo to provide the amino compound. A carboxylic acid drug can be converted to an ester (including silyl esters and thioesters), amide, or hydrazide prodrug, which be hydrolyzed in vivo to provide the carboxylic acid compound. Prodrugs for drugs that have functional groups different than those listed above are well known to the skilled artisan.

“Promoiety” refers to a group bonded to a drug, typically to a functional group of the drug, via bond(s) that are cleavable under specified conditions of use. The bond(s) between the drug and promoiety can be cleaved by enzymatic or non-enzymatic means. Under the conditions of use, for example following administration to a patient, the bond(s) between the drug and promoiety can be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation to which the prodrug is administered or the acidic conditions of the stomach, or the agent may be supplied exogenously. In certain embodiments of the present disclosure, the drug is gabapentin or pregabalin, and the promoiety is an acyloxyalkyloxycarbonyl group having the structure: 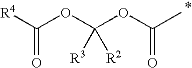
where R², R³, and R⁴ are defined herein.

“Protecting group” refers to a grouping of atoms that when attached to a reactive functional group in a molecule masks, reduces or prevents reactivity of the functional group. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (CBZ), tert-butoxycarbonyl (Boc), trimethylsilyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like. Examples of hydroxy protecting groups include, but are not limited to, those where the hydroxy group is either acylated or alkylated such as benzyl, and trityl ethers as well as alkyl ethers, tetrahydropyranyl ethers, trialkylsilyl ethers, and allyl ethers.

“Pharmaceutically acceptable solvate” refers to a molecular complex of a compound with one or more solvent molecules in a stoichiometric or non-stoichiometric amount. Such solvent molecules are those commonly used in the pharmaceutical art, which are known to be innocuous to recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water.

“Substituted” refers to a group in which one or more hydrogen atoms are independently replaced with the same or different substituent(s). Typical substituents include, but are not limited to, -M, —R⁶⁰, —O, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, and —C(NR⁶²)NR⁶⁰R⁶¹ where each M is independently a halogen; R⁶⁰, R⁶¹, R⁶², and R⁶³ are independently chosen from hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl, and substituted heteroaryl, or R⁶⁰ and R⁶¹ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, and —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, and —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, and —C(O)O⁻, where R⁶⁰, R⁶¹ and R⁶² are as defined above.

In certain embodiments, each substituent group is independently selected from halogen, —NH₂, —OH, —CN, —COOH, —C(O)NH₂, —C(O)OR⁵, and —NR⁵₃⁺, and each R⁵ is independently C₁₋₃ alkyl.

“Sustained release” refers to release of an agent from a dosage form at a rate effective to achieve a therapeutic or prophylactic amount of the agent, or active metabolite thereof, in the systemic blood circulation over a prolonged period of time relative to that achieved by oral administration of a conventional formulation of the agent. In some embodiments, release of the agent occurs over a period of at least 4 hours. In other embodiments, release of the agent occurs over a period of at least 8 hours. In still other embodiments, release of the agent occurs over a period of at least 12 hours.

“Tmax” is the time to the maximum concentration (Cmax) of a drug in the plasma or blood of a patient following administration of a dose of the drug or prodrug thereof to the patient.

“Treating” or “treatment” of a disease refers to arresting or ameliorating a disease, disorder, or at least one of the clinical symptoms of a disease or disorder. In certain embodiments, “treating” or “treatment” refers to arresting or ameliorating at least one physical parameter of the disease or disorder, which may or may not be discernible by the patient. In certain embodiments, “treating” or “treatment” refers to inhibiting or controlling the disease or disorder, either physically (e.g., stabilization of a discernible symptom), physiologically (e.g., stabilization of a physical parameter), or both. In certain embodiments, “treating” or “treatment” refers to delaying, in some cases indefinitely, the onset of a disease or disorder.

“Therapeutically effective amount” refers to the amount of a compound that, when administered to a subject for treating a disease, or at least one of the clinical symptoms of a disease, is sufficient to affect such treatment of the disease, disorder, or symptom. The “therapeutically effective amount” can vary depending, for example, on the compound, the disease, disorder, and/or symptoms of the disease, severity of the disease, disorder, and/or symptoms of the disease, the age, weight, and/or health of the patient to be treated, and the judgment of the prescribing physician. An appropriate amount in any given instance can be readily ascertained by those skilled in the art or capable of determination by routine experimentation.

“Vulvodynia” refers to chronic vulvar pain or discomfort in the absence of gross anatomic or neurological findings. The pain can be localized or generalized, and can last anywhere from a few months to several years. The pain or discomfort can include burning, soreness, rawness, throbbing, itching, stinging, parchedness, drying, swelling, and/or drawing sensations over the vulvar skin including the labia majora, labia minor, vestibule, etc., or only over certain parts of the vulvar skin as well as the rectal or anal skin. In addition, the pain or discomfort can include burning pain across the pubic line, shooting pains through the buttocks or thighs, and pain or numbness in other parts of the body. Vulvodynia includes vulvar vestibulitis in which pain is experienced in response to pressure on or stretching of the vestibule and dysesthetic vulvodynia in which vulvar pain is experienced as diffuse or generalized, is not necessarily limited to the vestibule, and is not necessarily induced by physical contact.

Reference is now be made in detail to certain embodiments of compounds and methods of the present disclosure. The disclosed embodiments are not intended to be limiting of the claims. To the contrary, the claims are intended to cover all alternatives, modifications, and equivalents.

### 4.2 GABA Analog Prodrugs

In some embodiments, a prodrug of a GABA analog is chosen from compounds of Formula (I) and Formula (II): 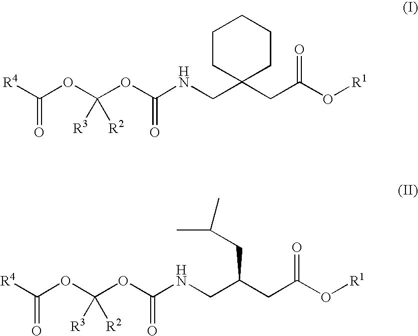
pharmaceutically acceptable salts thereof, pharmaceutically acceptable solvates of any of the foregoing, and pharmaceutically acceptable N-oxide of any of the foregoing, wherein:
R¹ is selected from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently selected from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or, R² and R³ together with the carbon atom to which they are bonded form a cycloalkyl, substituted cycloalkyl, cycloheteroalkyl, or substituted cycloheteroalkyl ring; and
R⁴ is selected from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments, for example, when R⁴ is substituted alkyl, each substituent group is independently selected from halogen, —NH₂, —OH, —CN, —COOH, —C(O)NH₂, —C(O)OR⁵, and —NR⁵₃⁺, and each R⁵ is independently C₁₋₃ alkyl.

In certain embodiments of compounds of Formulae (I) and (II), R¹ is hydrogen.

In certain embodiments of compounds of Formulae (I) and (II), R² and R³ are independently selected from hydrogen and C₁₋₆ alkyl.

In certain embodiments of compounds of Formulae (I) and (II), R³ is selected from methyl, ethyl, n-propyl, isopropyl, n-butyl, isobutyl, and sec-butyl, and R² is hydrogen.

In certain embodiments of compounds of Formulae (I) and (II), R³ is selected from methyl, ethyl, n-propyl, and isopropyl.

In certain embodiments of compounds of Formulae (I) and (II), R⁴ is selected from C₁₋₆ alkyl and C₁₋₆ substituted alkyl. In certain embodiments of compounds of Formulae (I) and (II) wherein R⁴ is selected from C₁₋₆ substituted alkyl, each substituent group is independently selected from halogen, —NH₂, —OH, —CN, —COOH, —C(O)NH₂, —C(O)OR⁵, and —NR⁵₃⁺, and each R⁵ is independently C₁₋₃ alkyl.

In certain embodiments of compounds of Formulae (I) and (II), R⁴ is selected from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formulae (I) and (II), R⁴ is selected from methyl, ethyl, n-propyl, isopropyl, n-butyl, and isobutyl.

In certain embodiments of compounds of Formulae (I) and (II), R¹ and R² is each hydrogen, R³ is C₁₋₆ alkyl, and R⁴ is selected from C₁₋₆ alkyl and C₁₋₆ substituted alkyl. In certain embodiments of compounds of Formulae (I) and (II), wherein R¹ and R² are each hydrogen, R³ is C₁₋₆ alkyl, and R⁴ is selected from C₁₋₆ substituted alkyl, each substituent group is independently selected from halogen, —NH₂, —OH, —CN, —COOH, —C(O)NH₂, —C(O)OR⁵, and —NR⁵₃⁺, and each R⁵ is independently C₁₋₃ alkyl.

In certain embodiments of compounds of Formulae (I) and (II), R¹ and R² are each hydrogen, R³ is selected from methyl, ethyl, n-propyl, isopropyl, n-butyl, isobutyl, and sec-butyl, and R⁴ is selected from methyl, ethyl, n-propyl, isopropyl, n-butyl, isobutyl, sec-butyl, n-pentyl, isopentyl, sec-pentyl, neopentyl, and 1,1-diethoxyethyl.

In certain embodiments of compounds of Formulae (I) and (II), R¹ and R² are each hydrogen, R³ is selected from methyl, ethyl, n-propyl, and isopropyl, and R⁴ is selected from methyl, ethyl, n-propyl, isopropyl, n-butyl, and isobutyl.

In certain embodiments, the compound of Formula (I) where R⁴ is isopropyl, R² is hydrogen, and R³ is methyl, is 1-{[(α-isobutanoyloxyethoxy)carbonyl]aminomethyl}-1-cyclohexane acetic acid or a pharmaceutically acceptable salt thereof, a pharmaceutically acceptable solvate of any of the foregoing, or a pharmaceutically acceptable N-oxide of any of the foregoing. In certain embodiments, the compound of Formula (I) where R⁴ is isopropyl, R² is hydrogen, and R³ is methyl, is a crystalline form of 1-{[(α-isobutanoyloxyethoxy)carbonyl]aminomethyl}-1-cyclohexane acetic acid as disclosed in Estrada et al., U.S. patent application Ser. No. 10/966,507. In certain embodiments, crystalline 1-{[(α-isobutanoyloxyethoxy)carbonyl]aminomethyl}-1-cyclohexane acetic acid has characteristic absorption peaks at 7.0°±0.3°, 8.2°±0.3°, 10.5°±0.3°, 12.8°±0.3°, 14.9°±0.3°, 16.4°±0.3°, 17.9°±0.3°, 18.1°±0.3°, 18.9°±0.3°, 20.9°±0.3°, 23.3°±0.3°, 25.3°±0.3°, and 26.6°±0.3° in an X-ray powder diffractogram. In certain embodiments, crystalline 1-{[(α-isobutanoyloxyethoxy)carbonyl]aminomethyl}-1-cyclohexane acetic acid has a melting point range from about 63° C. to about 64° C., and in certain embodiments, from about 64° C. to about 66° C.

Examples of compounds of Formula (I) include 1-{[(α-acetoxyethoxy)carbonyl]aminomethyl}-1-cyclohexane acetic acid, 1-{[(α-propanoyloxyethoxy)carbonyl]aminomethyl}-1-cyclohexane acetic acid, 1-{[(α-butanoyloxyethoxy)carbonyl]aminomethyl}-1-cyclohexane acetic acid, 1-{[(α-isobutanoyloxyethoxy)carbonyl]aminomethyl}-1-cyclohexane acetic acid, 1-{[(α-pivaloxyethoxy)carbonyl]aminomethyl}-1-cyclohexane acetic acid, 1-{[(α-acetoxymethoxy)carbonyl]aminomethyl}-1-cyclohexane acetic acid, 1-{[(α-propanoyloxymethoxy)carbonyl]aminomethyl}-1-cyclohexane acetic acid, 1-{[(α-butanoyloxymethoxy)carbonyl]aminomethyl}-1-cyclohexane acetic acid, 1-{[(α-isobutanoyloxymethoxy)carbonyl]aminomethyl}-1-cyclohexane acetic acid, 1-{[(α-pivaloxymethoxy)carbonyl]aminomethyl}-1-cyclohexane acetic acid, 1-{[(α-acetoxypropoxy)carbonyl]aminomethyl}-1-cyclohexane acetic acid, 1-{[(α-propanoyloxypropoxy)carbonyl]aminomethyl}-1-cyclohexane acetic acid, 1-{[(α-butanoyloxypropoxy)carbonyl]aminomethyl}-1-cyclohexane acetic acid, 1-{[(α-isobutanoyloxypropoxy)carbonyl]aminomethyl}-1-cyclohexane acetic acid, 1-{[(α-pivaloxypropoxy)carbonyl]aminomethyl}-1-cyclohexane acetic acid, 1-{[(α-acetoxyisopropoxy)carbonyl]aminomethyl}-1-cyclohexane acetic acid, 1-{[(α-propanoyloxyisopropoxy)carbonyl]aminomethyl}-1-cyclohexane acetic acid, 1-{[(α-butanoyloxyisopropoxy)carbonyl]aminomethyl}-1-cyclohexane acetic acid, 1-{[(α-isobutanoyloxyisopropoxy)carbonyl]aminomethyl}-1-cyclohexane acetic acid, 1-{[(α-pivaloxyisopropoxy)carbonyl]aminomethyl}-1-cyclohexane acetic acid, 1-{[(α-acetoxybutoxy)carbonyl]aminomethyl}-1-cyclohexane acetic acid, 1-{[(α-propanoyloxybutoxy)carbonyl]aminomethyl}-1-cyclohexane acetic acid 1-{[(α-butanoyloxybutoxy)carbonyl]aminomethyl}-1-cyclohexane acetic acid 1-{[(α-isobutanoyloxybutoxy)carbonyl]aminomethyl}-1-cyclohexane acetic acid, and 1-{[(α-pivaloxybutoxy)carbonyl]aminomethyl}-1-cyclohexane acetic acid, and pharmaceutically acceptable salts thereof, pharmaceutically acceptable solvates of any of the foregoing, or pharmaceutically acceptable N-oxides of any of the foregoing.

Examples of compounds of Formula (II) include 3-{[(α-acetoxyethoxy)carbonyl]aminomethyl}-5-methyl hexanoic acid, 3-{[(α-propanoyloxyethoxy)carbonyl]aminomethyl}-5-methyl hexanoic acid, 3-{[(α-butanoyloxyethoxy)carbonyl]aminomethyl}-5-methyl hexanoic acid, 3-{[(α-isobutanoyloxyethoxy)carbonyl]aminomethyl}-5-methyl hexanoic acid, 3-{[(α-pivaloxyethoxy)carbonyl]aminomethyl}-5-methyl hexanoic acid, 3-{[(α-acetoxymethoxy)carbonyl]aminomethyl}-5-methyl hexanoic acid, 3-{[(α-propanoyloxymethoxy)carbonyl]aminomethyl}-5-methyl hexanoic acid, 3-{[(α-butanoyloxymethoxy)carbonyl]aminomethyl}-5-methyl hexanoic acid, 3-{[(α-isobutanoyloxymethoxy)carbonyl]aminomethyl}-5-methyl hexanoic acid, 3-{[(α-pivaloxymethoxy)carbonyl]aminomethyl}-5-methyl hexanoic acid, 3-{[(α-acetoxypropoxy)carbonyl]aminomethyl}-5-methyl hexanoic acid, 3-{[(α-propanoyloxypropoxy)carbonyl]aminomethyl}-5-methyl hexanoic acid, 3-{[(α-butanoyloxypropoxy)carbonyl]aminomethyl}-5-methyl hexanoic acid, 3-{[(α-isobutanoyloxypropoxy)carbonyl]aminomethyl}-5-methyl hexanoic acid, 3-{[(α-pivaloxypropoxy)carbonyl]aminomethyl}-5-methyl hexanoic acid, 3-{[(α-acetoxyisopropoxy)carbonyl]aminomethyl}-5-methyl hexanoic acid, 3-{[(α-propanoyloxyisopropoxy)carbonyl]aminomethyl}-5-methyl hexanoic acid, 3-{[(α-butanoyloxyisopropoxy)carbonyl]aminomethyl}-5-methyl hexanoic acid, 3-{[(α-isobutanoyloxyisopropoxy)carbonyl]aminomethyl}-5-methyl hexanoic acid, 3-{[(α-pivaloxyisopropoxy)carbonyl]aminomethyl}-5-methyl hexanoic acid, 3-{[(α-acetoxybutoxy)carbonyl]aminomethyl}-5-methyl hexanoic acid, 3-{[(α-propanoyloxybutoxy)carbonyl]aminomethyl}-5-methyl hexanoic acid, 3-{[(α-butanoyloxybutoxy)carbonyl]aminomethyl}-5-methyl hexanoic acid, 3-{[(α-isobutanoyloxybutoxy)carbonyl]aminomethyl}-5-methyl hexanoic acid, and 3{[(α-pivaloxybutoxy)carbonyl]aminomethyl}-5-methyl hexanoic acid, and pharmaceutically acceptable salts thereof, pharmaceutically acceptable solvates of any of the foregoing, or pharmaceutically acceptable N-oxides of any of the foregoing.

In certain embodiments, the compound of Formula (II) is 3-{[(α-isobutanoyloxyethoxy)carbonyl]aminomethyl}-5-methyl hexanoic acid or a pharmaceutically acceptable salt thereof, a pharmaceutically acceptable solvate of any of the foregoing, or a pharmaceutically acceptable N-oxide of any of the foregoing.

### 4.3 Methods of Synthesis of Prodrugs of GABA Analogs

Methods of synthesizing prodrugs of GABA analogs, including methods of synthesizing compounds of Formula (I) and Formula (II) are disclosed in Gallop et al., International Publication No. WO 02/100347, Gallop et al., United States Application Publication No. 2004/0077553, and Bhat et al., United States Application Publication No. 2005/0070715. Other methods for synthesis of prodrugs of GABA analogs have also been disclosed (see Bryans et al., International Publication No. WO 01/90052; U.K. Application GB 2,362,646; European Applications EP 1,201,240 and 1,178,034; Yatvin et al., U.S. Pat. No. 6,024,977; Gallop et al., International Publication No. WO 02/28881; Gallop et al., International Publication No. WO 02/28883; Gallop et al., International Publication No. WO 02/28411; Gallop et al., International Publication No. WO 02/32376; Gallop et al., International Publication No. WO 02/42414).

### 4.4 Therapeutic Methods of Use

In certain embodiments, a compound of Formula (I) or Formula (II) or a pharmaceutical composition thereof can be administered to a patient suffering from vulvodynia. The suitability of a compound of Formula (I), Formula (II) or pharmaceutical compositions thereof to treat vulvodynia can be determined by methods known to the skilled artisan. The patient is a mammal, for example a human. The patient may be a female of any age.

The compounds disclosed herein, for example the gabapentin prodrug 1-{[(α-isobutanoyloxyethoxy)carbonyl]aminomethyl}-1-cyclohexane acetic acid, may be more efficacious than the parent drug molecule (e.g., gabapentin or pregabalin) in treating vulvodynia because the disclosed compounds require less time to reach a therapeutic concentration in the systemic circulation, i.e., the compounds disclosed herein have a shorter Tₘₐₓ than their parent drug counterparts when taken orally. For example, an immediate release formulation of the prodrug 1-{[(α-isobutanoyloxyethoxy)carbonyl]aminomethyl}-1-cyclohexane acetic acid provides a Tₘₐₓ within from about 2 hours to about 2.6 hours following oral administration compared from about 2.8 to about 3.3 hours for an equivalent immediate release formulation of the parent drug, gabapentin.

The compounds disclosed herein, for example, the gabapentin prodrug 1-{[(α-isobutanoyloxyethoxy)carbonyl]aminomethyl}-1-cyclohexane acetic acid, is more efficacious than the parent drug molecule (i.e., gabapentin or pregabalin) in treating vulvodynia because the disclosed compounds provide a lower Cₘₐₓ while maintaining a similar AUC compared to their gabapentin and pregabalin counterparts when taken orally. This provides the advantage over the parent drugs (i.e., gabapentin or pregabalin) of reducing the potential for adverse side effects associated with high plasma gabapentin or pregabalin concentrations while maintaining a therapeutically effective concentration of the drugs for treating vulvodynia. It is believed that the compounds disclosed herein, for example, the gabapentin prodrug 1-{[(α-isobutanoyloxyethoxy)carbonyl]aminomethyl}-1-cyclohexane acetic acid, are absorbed from the gastrointestinal lumen into the blood by a different mechanism than that by which gabapentin and other known GABA analogs are absorbed. For example, gabapentin is believed to be actively transported across the gut wall by a carrier transporter localized in the human small intestine. The gabapentin transporter is easily saturated, which means that the amount of gabapentin absorbed into the blood may not be proportional to the amount of gabapentin that is administered orally, since once the transporter is saturated, further absorption of gabapentin does not occur to any significant degree. In comparison to gabapentin, the compounds disclosed herein, for example, the gabapentin prodrug 1-{[(α-isobutanoyloxyethoxy)carbonyl]aminomethyl}-1-cyclohexane acetic acid, do not exhibit saturated absorption in the small intestine over a therapeutically effective dosage range and are believed to be absorbed across the gut wall along a greater portion of the gastrointestinal tract, including the colon.

Because the compounds disclosed herein can be formulated in sustained release formulations, which provide for sustained release of a compound of Formula (I) or Formula (II) into the gastrointestinal tract, for example, within the colon, over a period of hours, the compounds, such as the gabapentin prodrug 1-{[(α-isobutanoyloxyethoxy)carbonyl]aminomethyl}-1-cyclohexane acetic acid, may be more efficacious than their respective parent drugs (e.g., gabapentin or pregabalin) in treating vulvodynia. The ability of the compounds of the present disclosure to be used in sustained release oral dosage forms can reduce the dosing frequency necessary for maintenance of a therapeutically effective drug concentration in the systemic circulation.

### 4.5 Therapeutic/Prophylactic Administration

Dosage forms comprising compound of Formula (I) or Formula (II) may be used to treat vulvodynia. The dosage forms may be administered or applied singly or in combination with other agents. The dosage forms may also deliver a compound of Formula (I) or Formula (II) to a patient in combination with another pharmaceutically active agent including another prodrug of a GABA analog and/or another active agent known or believed to be capable of treating vulvodynia.

When used in the present methods of treatment, upon releasing a compound of Formula (I) or Formula (II) analog in vivo, the dosage forms can provide the corresponding GABA analog, e.g., gabapentin or pregabalin, in the systemic circulation of a patient. The promoiety or promoieties of the prodrug may be cleaved either chemically and/or enzymatically. One or more enzymes present in the stomach, intestinal lumen, intestinal tissue, blood, liver, brain, or any other suitable tissue of a mammal may cleave the promoiety or promoieties of the prodrug. The mechanism of cleavage is not critical to the disclosed methods. In certain embodiments, the GABA analog that is formed by cleavage of the promoiety from the prodrug does not contain substantial quantities of lactam contaminant (such as, less than about 0.5% by weight, for example, less than about 0.2% by weight, and in certain embodiments, less than about 0.1% by weight) for the reasons described in Augart et al., U.S. Pat. No. 6,054,482. The extent of release of lactam contaminant from the prodrugs may be assessed using standard in vitro analytical methods.

Some therapeutically effective GABA analogs, e.g., gabapentin and pregabalin, have poor passive permeability across the gastrointestinal mucosa, probably because of their zwitterionic character at physiological pH. Gabapentin, pregabalin, and other GABA analogs are actively transported across the gastrointestinal tract by one or more amino acid transporters (e.g., the “large neutral amino acid transporter”). However, the large neutral amino acid transporter is expressed predominantly within cells lining the lumen of a limited region of the small intestine, which provides a limited window for drug absorption and leads to an overall dose-dependent drug bioavailability that decreases with increasing dose.

In certain embodiments, a compound of Formula (I) or Formula (II) can be suitable for oral administration. In certain embodiments, the promoiety or promoieties are cleaved after absorption of the compound of Formula (I) or Formula (II) by the gastrointestinal tract (e.g., in intestinal tissue, blood, liver or other suitable tissue of the patient) following oral administration of a compound of Formula (I) or Formula (II). The promoiety or promoieties may make the prodrug a substrate for one or more transporters expressed in the large intestine (i.e., colon), and/or, for GABA analogs that are poorly absorbed across the gastrointestinal mucosa (e.g., gabapentin and pregabalin), may facilitate the ability of the prodrug to be passively absorbed across the gastrointestinal mucosa.

### 4.6 Pharmaceutical Compositions

Pharmaceutical compositions disclosed herein comprise a therapeutically effective amount of one or more GABA analog prodrugs of Formulae (I) and/or (II), together with a suitable amount of a pharmaceutically acceptable vehicle, so as to provide a form for proper administration to a patient. In certain embodiments, the one or more compounds of Formula (I) and/or Formula (II) are in a purified form. When administered to a patient, the prodrug and pharmaceutically acceptable vehicles may be sterile. Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. Other examples of suitable pharmaceutical vehicles have been described in the art (see Remington's Pharmaceutical Sciences, Philadelphia College of Pharmacy and Science, 19^{th} Edition, 1995). Compositions of the present disclosure, if desired, can also contain minor amounts of wetting or emulsifying agents, or pH buffering agents. In addition, auxiliary, stabilizing, thickening, lubricating, and coloring agents can be included.

Pharmaceutical compositions can be manufactured, for example, by means of conventional mixing, dissolving, granulating, dragee-making, levigating, emulsifying, encapsulating, entrapping, or lyophilizing processes. Pharmaceutical compositions can be formulated in a conventional manner using one or more physiologically acceptable carriers, diluents, excipients, or auxiliaries, which facilitate processing of compounds disclosed herein into preparations that can be used pharmaceutically. Proper formulation is dependent upon the route of administration chosen.

The present pharmaceutical compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In some embodiments, the pharmaceutically acceptable vehicle can be a capsule (see e.g., Grosswald et al., U.S. Pat. No. 5,698,155). In some embodiments, compositions can be formulated for oral delivery, for example, for oral sustained release administration. In certain embodiments, compositions can be formulated for topical delivery, and in certain embodiments, for topical sustained release administration.

Pharmaceutical compositions for oral delivery can be, for example, in the form of tablets, lozenges, aqueous or oily suspensions, granules, powders, emulsions, capsules, syrups, or elixirs. Orally administered compositions can contain one or more optional agents, for example, sweetening agents such as fructose, aspartame, or saccharin, flavoring agents such as peppermint, oil of wintergreen, or cherry coloring agents, and preserving agents, to provide a pharmaceutically palatable preparation. Moreover, when in tablet or pill form, the compositions can be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, and the like. Such vehicles may be of pharmaceutical grade.

For oral liquid preparations such as, for example, suspensions, elixirs, and solutions; suitable carriers, excipients, or diluents include water, saline, alkyleneglycols (e.g., propylene glycol), polyalkylene glycols (e.g., polyethylene glycol), oils, alcohols, slightly acidic buffers ranging from about pH 4 to about pH 6 (e.g., acetate, citrate, ascorbate ranging from about 5 mM to about 50 mM), and the like. Additionally, flavoring agents, preservatives, coloring agents, bile salts, acylcarnitines, and the like can be added.

For topical formulations of GABA analog prodrugs of Formulae (I) and (II), creams, gels, or viscous lotions can be used as appropriate delivery forms. Such formulations can comprise one or more GABA analog prodrugs of Formulae (I) and/or (II), which can be in purified form, together with a suitable amount of a pharmaceutically acceptable topical vehicle including, but not limited to, gels, lotions, creams, ointments, and liquids.

Compositions for topical administration include those for delivery via the organs comprising the vulva (vulval), the vestibule (vestibular), the vagina (vaginal), and through the skin (dermal). Topical delivery systems also include transdermal patches containing at least one GABA analog prodrugs of Formulae (I) and/or (II) to be administered. Delivery through the skin can be achieved by diffusion or by more active energy sources such as iontophoresis or electrotransport.

Compositions for topical administration to the skin include ointments, creams, gels, patches, pastes, and sprays comprising at least one GABA analog prodrugs of Formulae (I) and/or (II), to be administered in a pharmaceutical acceptable vehicle. Formulations of a GABA analog prodrug of Formulae (I) and/or (II), for topical use, such as in creams, ointments, and gels, can include an oleaginous or water-soluble ointment base. For example, topical compositions can include vegetable oils, animal fats, and in certain embodiments, semisolid hydrocarbons obtained from petroleum. Topical compositions can further include white ointment, yellow ointment, cetyl esters wax, oleic acid, olive oil, paraffin, petrolatum, white petrolatum, spermaceti, starch glycerite, white wax, yellow wax, lanolin, anhydrous lanolin, and glyceryl monostearate. Various water-soluble ointment bases can also be used, including glycol ethers and derivatives, polyethylene glycols, polyoxyl 40 stearate, and polysorbates.

Compositions suitable for vaginal administration can be provided as pessaries, tampons, creams, gels, pastes, foams, or spray formulations containing in addition to at least one GABA analog prodrug of Formulae (I) and/or (II) such vehicles as are known in the art to be appropriate. When a GABA analog prodrug of Formulae (I) or (II) is acidic, it can be included in any of the above-described formulations as the free acid, a pharmaceutically acceptable salt, a solvate, or an N-oxide thereof. Pharmaceutically acceptable salts can substantially retain the activity of the free acid, may be prepared by reaction with bases, and can be more soluble in aqueous and other protic solvents than the corresponding free acid form.

In certain embodiments, pharmaceutical compositions of the present disclosure contain no or only low levels of lactam side products formed by intramolecular cyclization of the GABA analog and/or GABA analog prodrug of Formula (I) or Formula (II). In certain embodiments, the compositions are stable to extended storage (for example, greater than one year) without substantial lactam formation (for example, less than about 0.5% lactam by weight, such as, less than about 0.2% lactam by weight, and in certain embodiments, less than about 0.1% lactam by weight).

Pharmaceutical compositions provided by the present disclosure may be provided as immediate release or sustained release formulations.

### 4.7 Sustained Release Oral Dosage Forms

Methods that involve oral administration of a compound of Formula (I) or Formula (II) to treat vulvodynia can be practiced with a number of different dosage forms, which provide sustained release of the prodrug. Such sustained release oral dosage forms are used for administering those compounds of Formula (I) or Formula (II) that are absorbed by cells lining the large intestine since these dosage forms are generally well adapted to deliver a prodrug to that location of the gastrointestinal tract.

In some embodiments, the dosage form can comprise beads, which on dissolution or diffusion release the prodrug over an extended period of hours, such as, over a period of at least 4 hours, for example, over a period of at least about 8 hours and in certain embodiments, over a period of at least 12 hours. The prodrug-releasing beads can have a central composition or core comprising a prodrug and at least one pharmaceutically acceptable vehicle, including optional lubricants, antioxidants, and buffers. The beads can be medical preparations with a diameter ranging from about 1 to about 2 mm. Individual beads can comprise doses of a compound of the present disclosure, for example, doses of up to about 40 mg of the prodrug. The beads, in some embodiments, can be formed of non-crosslinked materials to enhance their discharge from the gastrointestinal tract. The beads can be coated with a release rate-controlling polymer that gives a time-release profile such as a pH independent or pH dependent release coating.

The time-release beads can be manufactured into a tablet for therapeutically effective prodrug administration. The beads can be made into matrix tablets by direct compression of a plurality of beads coated with, for example, an acrylic resin and blended with excipients such as hydroxypropylmethyl cellulose. The manufacture of beads has been disclosed in the art (Lu, Int. J. Pharm. 1994, 112, 117-124; Pharmaceutical Sciences by Remington, 14^{th} ed, pp 1626-1628 (1970); Fincher, J. Pharm. Sci. 1968, 57, 1825-1835; Benedikt, U.S. Pat. No. 4,083,949), as has the manufacture of tablets (Pharmaceutical Sciences, by Remington, 17^{th} Ed, Ch. 90, pp 1603-1625 (1985)).

One type of sustained release oral dosage formulation that can be used with compounds of the present disclosure comprises an inert core, such as a sugar sphere, coated with an inner drug-containing layer and an outer membrane layer controlling drug release from the inner layer. A “seal coat” can be provided between the inert core and the layer containing the active ingredient. When the core is of a water-soluble or water-swellable inert material, the seal coat can be in the form of a relatively thick layer of a water-insoluble polymer. Such a controlled release bead can thus comprise (i) a core unit of a substantially water-soluble or water-swellable inert material, (ii) a first layer on the core unit of a substantially water-insoluble polymer, (iii) a second layer covering the first layer and containing an active ingredient, and (iv) a third layer on the second layer of polymer effective for controlled release of the active ingredient, wherein the first layer is adapted to control water penetration into the core.

Usually, the first layer (ii) above can constitute more than about 2% (w/w) of the final bead composition, such as, more than about 3% (w/w), e.g., from about 3% to about 80% (w/w). The amount of the second layer (ii) above can constitute from about 0.05% to about 60% (w/w), such as from about 0.1% to about 30% (w/w) of the final bead composition. The amount of the third layer (iv) above can constitute from about 1% to about 50% (w/w), such as, from about 2% to about 25% (w/w) of the final bead composition. The core unit can have a size ranging from about 0.05 mm to about 2 mm. The controlled release beads can be provided in a multiple unit formulation, such as a capsule or a tablet.

The cores can comprise a water-soluble or swellable material and can be any such material that is conventionally used as cores or any other pharmaceutically acceptable water-soluble or water-swellable material made into beads or pellets. The cores can be spheres of materials such as sucrose/starch (Sugar Spheres NF), sucrose crystals, or extruded and dried spheres typically comprising excipients such as microcrystalline cellulose and lactose. The substantially water-insoluble material in the first, or seal coat layer can be a “GI insoluble” or “GI partially insoluble” film forming polymer dispersed or dissolved in a solvent. Examples include, but are not limited to, ethyl cellulose, cellulose acetate, cellulose acetate butyrate, polymethacrylates such as ethyl acrylate/methyl methacrylate copolymer (Eudragit NE-30-D), ammonio methacrylate copolymer types A and B (Eudragit RL30D and RS30D), and silicone elastomers. In certain embodiments, a plasticizer can be used together with the polymer. Examples of plasticizers include, but are not limited to, dibutylsebacate, propylene glycol, triethylcitrate, tributylcitrate, castor oil, acetylated monoglycerides, acetyl triethylcitrate, acetyl butylcitrate, diethyl phthalate, dibutyl phthalate, triacetin, and fractionated coconut oil (medium-chain triglycerides). The second layer containing the active ingredient can comprise an active ingredient with or without a polymer as a binder. The binder can be hydrophilic, and in certain embodiments, can be water-soluble or water-insoluble. Examples of polymers that can be used in the second layer containing the active drug include hydrophilic polymers such as, for example, polyvinylpyrrolidone (PVP), polyalkylene glycol such as polyethylene glycol, gelatine, polyvinyl alcohol, starch, and derivatives thereof, cellulose derivatives such as hydroxypropylmethyl cellulose (HPMC), hydroxypropyl cellulose, carboxymethyl cellulose, methyl cellulose, ethyl cellulose, hydroxyethyl cellulose, carboxyethyl cellulose, and carboxymethylhydroxyethyl cellulose, acrylic acid polymers, and polymethacrylates. The ratio of drug to hydrophilic polymer in the second layer can range from about 1:100 to about 100:1 (w/w). Suitable polymers for use in the third layer, or membrane, for controlling the drug release can be selected from water-insoluble polymers or polymers with pH-dependent solubility, such as, for example, ethyl cellulose, hydroxypropylmethyl cellulose phthalate, cellulose acetate phthalate, cellulose acetate trimellitate, polymethacrylates, or mixtures thereof, optionally combined with plasticizers, such as those mentioned above. Optionally, the controlled release layer comprises, in addition to the polymers above, other substance(s) with different solubility characteristics to adjust the permeability and thereby the release rate of the controlled release layer. Example of polymers that can be used as a modifier together with, for example, ethyl cellulose include, but are not limited to, HPMC, hydroxyethyl cellulose, hydroxypropyl cellulose, methylcellulose, carboxymethylcellulose, polyethylene glycol, polyvinylpyrrolidone (PVP), polyvinyl alcohol, polymers with pH-dependent solubility such as cellulose acetate phthalate or ammonio methacrylate copolymer, methacrylic acid copolymer, and mixtures thereof. Additives such as sucrose, lactose, and pharmaceutical grade surfactants can also be included in the controlled release layer, if desired.

Preparation of the multiple unit formulation can comprise the additional step of transforming the prepared beads into a pharmaceutical formulation, such as by filling a predetermined amount of the beads into a capsule, or compressing the beads into tablets. Examples of multi-particulate sustained release oral dosage forms are described in, for example, U.S. Pat. Nos. 6,627,223 and 5,229,135.

In some embodiments, an oral sustained release pump may be used (Langer, supra; Sefton, CRC Crit Ref Biomed. Eng. 1987, 14, 201; Saudek et al., N. Engl. J Med. 1989, 321, 574).

In other embodiments, polymeric materials can be used (see “Medical Applications of Controlled Release,” Langer and Wise (eds.), CRC Press., Boca Raton, Fla. (1974); “Controlled Drug Bioavailability,” Drug Product Design and Performance, Smolen and Ball (eds.), Wiley, N.Y. (1984); Langer et al., J Macromol. Sci. Rev. Macromol Chem. 1983, 23, 61; Levy et al., Science, 1985, 228, 190; During et al., Ann. Neurol. 1989, 25, 351; Howard et al., J. Neurosurg. 1989, 71, 105). In certain embodiments, polymeric materials are used for oral sustained release delivery. Polymers for oral sustained release delivery include sodium carboxymethylcellulose, hydroxypropylcellulose, hydroxypropylmethylcellulose, and hydroxyethylcellulose, for example hydroxypropylmethylcellulose. Other cellulose ethers have been described (Alderman, Int. J. Pharm. Tech. & Prod. Mfr. 1984, 5(3), 1-9). Factors affecting drug release are well known to the skilled artisan and have been described in the art (Bamba et al., Int. J. Pharm. 1979, 2, 307).

In some embodiments, enteric-coated preparations can be used for oral sustained release administration. Examples of polymers useful in enteric-coated preparations include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release), and polymers that form firm layers that are destroyed by an increase in pressure (i.e., pressure-controlled release).

In certain embodiments, drug-releasing lipid matrices can be used for oral sustained release administration. One example is when solid microparticles of the prodrug are coated with a thin controlled release layer of a lipid (e.g., glyceryl behenate and/or glyceryl palmitostearate) as disclosed in Farah et al., U.S. Pat. No. 6,375,987 and Joachim et al., U.S. Pat. No. 6,379,700. The lipid-coated particles can optionally be compressed to form a tablet. Another controlled release lipid-based matrix material which is suitable for sustained release oral administration comprises polyglycolized glycerides as disclosed in Roussin et al., U.S. Pat. No. 6,171,615.

In certain embodiments, prodrug-releasing waxes can be used for oral sustained release administration. Examples of suitable sustained prodrug-releasing waxes are disclosed in Cain et al., U.S. Pat. No. 3,402,240 (carnauba wax, candelilla wax, esparto wax, and ouricury wax); Shtohryn et al., U.S. Pat. No. 4,820,523 (hydrogenated vegetable oil, bees wax, carnauba wax, paraffin, candelilla, ozokerite, and mixtures thereof); and Walters, U.S. Pat. No. 4,421,736 (mixture of paraffin and castor wax).

In certain embodiments, osmotic delivery systems can be used for oral sustained release administration (Verma et al., Drug Dev. Ind. Pharm. 2000, 26, 695-708). In certain embodiments, OROS® systems made by Alza Corporation, Mountain View, Calif. can be used for oral sustained release delivery devices (Theeuwes et al., U.S. Pat. No. 3,845,770; Theeuwes et al., U.S. Pat. No. 3,916,899).

In certain embodiments, a controlled-release system can be placed in proximity of the target of the compound of Formula (I) or Formula (II), thus requiring only a fraction of the systemic dose (see e.g., Goodson, in “Medical Applications of Controlled Release,” supra, vol. 2, pp. 115-138 (1984)). Other controlled-release systems, discussed in Langer, Science 1990, 249, 1527-1533, can also be used.

In certain embodiments, the dosage form can comprise a compound of Formula (I) or Formula (II) coated on a polymer substrate. The polymer can be an erodible, or a nonerodible polymer. The coated substrate can be folded onto itself to provide a bilayer polymer drug dosage form. For example, a compound of Formula (I) or Formula (II) can be coated onto a polymer such as a polypeptide, collagen, gelatin, polyvinyl alcohol, polyorthoester, polyacetyl, or a polyorthocarbonate and the coated polymer folded onto itself to provide a bilaminated dosage form. In operation, a bioerodible dosage form erodes at a controlled rate to dispense a compound over a sustained release period. Examples of biodegradable polymers include biodegradable poly(amides), poly(amino acids), poly(esters), poly(lactic acid), poly(glycolic acid), poly(carbohydrate), poly(orthoester), poly(orthocarbonate), poly(acetyl), poly(anhydrides), biodegradable poly(dihydropyrans), and poly(dioxinones), which are known in the art (Rosoff, Controlled Release of Drugs, Chap. 2, pp. 53-95 (1989); Heller et al., U.S. Pat. No. 3,811,444; Michaels, U.S. Pat. No. 3,962,414; Capozza, U.S. Pat. No. 4,066,747; Schmitt, U.S. Pat. No. 4,070,347; Choi et al., U.S. Pat. No. 4,079,038; Choi et al., U.S. Pat. No. 4,093,709).

In certain embodiments, the dosage form can comprise a prodrug loaded into a polymer such as a prodrug-releasing polymer that releases the prodrug by diffusion through a polymer, by flux through pores, or by rupture of a polymer matrix. The drug delivery polymeric dosage form can comprise from about 10 mg to about 2500 mg of a prodrug homogenously contained in or on a polymer. The dosage form can comprise at least one exposed surface at the beginning of dose delivery. The non-exposed surface, when present, can be coated with a pharmaceutically acceptable material impermeable to the passage of a prodrug. The dosage form can be manufactured by procedures known in the art. An example of providing a dosage form comprises blending a pharmaceutically acceptable carrier such as polyethylene glycol with a known dose of prodrug at an elevated temperature, (e.g., 37° C.), and adding the blended composition to a silastic medical grade elastomer with a cross-linking agent, for example, octanoate, followed by casting in a mold. The step can be repeated for each optional successive layer. The system can be allowed to set for about 1 hour to provide the dosage form. Representative polymers for manufacturing the dosage form include olefins, vinyl polymers, addition polymers, condensation polymers, carbohydrate polymers, and silicone polymers as represented by polyethylene, polypropylene, polyvinyl acetate, polymethylacrylate, polyisobutylmethacrylate, polyalginate, polyamide, and polysilicone. The polymers, and procedures for manufacturing the polymers, have been described in the art (Coleman et al., Polymers 1990, 31, 1187-1231; Roerdink et al., Drug Carrier Systems 1989, 9, 57-10; Leong et al., Adv. Drug Delivery Rev. 1987, 1, 199-233; Roff et al., Handbook of Common Polymers, 1971, CRC Press; Chien et al., U.S. Pat. No. 3,992,518).

In certain embodiments, the dosage form can comprise a plurality of pills. Time-release pills can provide a number of individual doses for providing various time doses for achieving a sustained-release prodrug delivery profile over an extended period of time such as, for example, up to about 24 hours. The matrix of the time-release pills can comprise a hydrophilic polymer such as, for example, polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrophilic matrix can comprise a plurality of 4 to 50 pills, each pill comprising, for example, a dose population of from about 10 ng, about 0.5 mg, about 1 mg, about 1.2 mg, about 1.4 mg, about 1.6 mg, about 5.0 mg, etc. The pills can comprise a release rate-controlling wall ranging from about 0.001 mm to about 10 mm thick to provide for the timed release of prodrug. Representative wall-forming materials include a triglyceryl ester such as glyceryl tristearate, glyceryl monostearate, glyceryl dipalmitate, glyceryl laureate, glyceryl didecenoate, and glyceryl tridenoate. Other useful wall forming materials include polyvinyl acetate, phthalate, methylcellulose phthalate, and microporous olefins. Procedures for manufacturing time-release pills are disclosed in Urquhart et al., U.S. Pat. No. 4,434,153; Urquhart et al., U.S. Pat. No. 4,721,613; Theeuwes, U.S. Pat. No. 4,853,229; Barry, U.S. Pat. No. 2,996,431; Neville, U.S. Pat. No. 3,139,383; Mehta, U.S. Pat. No. 4,752,470.

In certain embodiments, the dosage form can comprise an osmotic dosage form, which comprises a semipermeable wall that surrounds a therapeutic composition comprising the prodrug. In use within a patient, an osmotic dosage form comprising a homogenous composition, imbibes fluid through the semipermeable wall into the dosage form in response to the concentration gradient across the semipermeable wall. The therapeutic composition in the dosage form develops an osmotic pressure differential that causes the therapeutic composition to be administered through an exit from the dosage form over a prolonged period of time such as, for example, up to about 24 hours, and in some embodiments, up to about 30 hours, to provide controlled and sustained prodrug release. These delivery platforms can provide a zero order or an essentially zero order delivery profile, as opposed to the spiked profiles characteristic of immediate release formulations.

In certain embodiments, the dosage form can comprise another osmotic dosage form comprising a wall surrounding a compartment, the wall comprising a semipermeable polymeric composition permeable to the passage of fluid and substantially impermeable to the passage of prodrug present in the compartment, a prodrug-containing layer composition in the compartment, a hydrogel push layer composition in the compartment comprising an osmotic formulation for imbibing and absorbing fluid for expanding in size for pushing the prodrug composition layer from the dosage form, and at least one passageway in the wall for releasing the prodrug composition. The osmotic dosage form delivers the prodrug by imbibing fluid through the semipermeable wall at a fluid imbibing rate determined by the permeability of the semipermeable wall and the osmotic pressure across the semipermeable wall causing the push layer to expand, thereby delivering the prodrug from the dosage form through the exit passageway to a patient over a prolonged period of time such as, for example, up to about 24 or even up to about 30 hours. The hydrogel layer composition can comprise from about 10 mg to about 1000 mg of a hydrogel such as a polyalkylene oxide of from about 1,000,000 to about 8,000,000 weight-average molecular weight, or from about 10 mg to about 1000 mg of an alkali carboxymethylcellulose of from about 10,000 to about 6,000,000 weight-average molecular weight such as sodium carboxymethylcellulose or potassium carboxymethylcellulose. The hydrogel expansion layer can comprise from about 0 mg to about 350 mg, and include from about 0.1 mg to about 250 mg of a hydroxyalkylcellulose ranging from about 7,500 to about 4,500,00 weight-average molecular weight (e.g., hydroxymethylcellulose, hydroxyethylcellulose, hydroxypropylcellulose, hydroxybutylcellulose or hydroxypentylcellulose), from about 1 mg to about 50 mg of an osmagent selected from sodium chloride, potassium chloride, potassium acid phosphate, tartaric acid, citric acid, raffinose, magnesium sulfate, magnesium chloride, urea, inositol, sucrose, glucose, and sorbitol, from about 0 to about 5 mg of a colorant, such as ferric oxide; from about 0 mg to about 30 mg of a hydroxypropylalkylcellulose of from about 9,000 to about 225,000 average-number molecular weight selected from hydroxypropylethylcellulose, hydroxypropylpentylcellulose, hydroxypropylmethylcellulose, and hydropropylbutylcellulose, from about 0.00 to about 1.5 mg of an antioxidant selected from ascorbic acid, butylated hydroxyanisole, butylated hydroxyquinone, butylhydroxyanisole, hydroxycomarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin, and ethanolamine, and from about 0 mg to about 7 mg of a lubricant selected, for example, from calcium stearate, magnesium stearate, zinc stearate, magnesium oleate, calcium palmitate, sodium suberate, potassium laurate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid.

In the osmotic dosage forms, the semipermeable wall can comprise a composition that is permeable to the passage of fluid and impermeable to the passage of prodrug. The wall can be nontoxic and can comprise a polymer such as cellulose acrylate, cellulose diacylate, cellulose triacylate, cellulose acetate, cellulose diacetate, or cellulose triacetate. The wall can comprise about 75 wt % (weight percent) to about 100 wt % of the cellulosic wall-forming polymer; or, the wall can comprise additionally about 0.01 wt % to about 80 wt % of polyethylene glycol, or about 1 wt % to about 25 wt % of a cellulose ether selected from hydroxypropylcellulose or a hydroxypropylalkycellulose such as hydroxypropylmethylcellulose. The total weight percent of all components comprising the wall is equal to 100 wt %. The internal compartment comprises the prodrug-containing composition alone or in layered position with an expandable hydrogel composition. The expandable hydrogel composition in the compartment can increase in dimension by imbibing the fluid through the semipermeable wall, causing the hydrogel to expand and occupy space in the compartment, whereby the drug composition is pushed from the dosage form. The therapeutic layer and the expandable layer act together during the operation of the dosage form for the release of prodrug to a patient over time. The dosage form can comprise a passageway in the wall that connects the exterior of the dosage form with the internal compartment. The osmotic dosage form can be designed to deliver prodrug from the dosage form to the patient at a zero order rate of release over a period of up to about 24 hours.

The expression “passageway” as used herein comprises means and methods suitable for the metered release of the prodrug from the compartment of the dosage form. The exit means can comprise at least one passageway, including an orifice, bore, aperture, pore, porous element, hollow fiber, capillary tube, channel, porous overlay, or porous element that provides for the osmotic controlled release of prodrug. The passageway can include a material that erodes or is leached from the wall in a fluid environment of use to produce at least one controlled-release dimensioned passageway. Materials suitable for forming a passageway, or a multiplicity of passageways include, for example, a leachable poly(glycolic) acid or poly(lactic) acid polymer in the wall, a gelatinous filament, poly(vinyl alcohol), leachable polysaccharides, salts, and oxides. A pore passageway, or more than one pore passageway, can be formed by leaching a leachable compound, such as sorbitol, from the wall. The passageway possesses controlled-release dimensions, such as round, triangular, square, or elliptical, for the metered release of prodrug from the dosage form. The dosage form can be constructed with one or more passageways in spaced apart relationship on a single surface or on more than one surface of the wall. The expression “fluid environment” denotes an aqueous or biological fluid as in a human patient, including the gastrointestinal tract. Passageways and equipment for forming passageways are disclosed in Theeuwes et al., U.S. Pat. No. 3,845,770; Theeuwes et al., U.S. Pat. No. 3,916,899; Saunders et al., U.S. Pat. No. 4,063,064; Theeuwes et al., U.S. Pat. No. 4,088,864 and Ayer et al., U.S. Pat. No. 4,816,263. Passageways formed by leaching are disclosed in Ayer et al., U.S. Pat. No. 4,200,098 and Ayer et al., U.S. Pat. No. 4,285,987.

Examples of sustained release oral dosage forms of GABA analogs are disclosed in Cundy et al., U.S Application Publication No. 2004/0198820 and Cundy et al., U.S. Application Publication No. 2006/0141034.

Regardless of the specific sustained release oral dosage form used, the prodrug can be released from the dosage form over a period of at least about 4 hours, such as, over a period of at least about 8 hours, and in certain embodiments, over a period of at least about 12 hours. Further, in certain embodiments, the dosage form releases from about 0% to about 20% of the prodrug in about 0 to about 2 hours, from about 20% to about 50% of the prodrug in about 2 to about 12 hours, from about 50% to about 85% of the prodrug in about 3 to about 20 hours and greater than about 75% of the prodrug in about 5 to about 18 hours. The sustained release oral dosage form can provide a concentration of gabapentin or pregabalin in the systemic circulation of a patient over time, which curve has an area under the curve (AUC) that is proportional to the dose of the compound of Formula (I) or Formula (II) respectively, administered, and a maximum concentration Cₘₐₓ. In certain embodiments, the Cₘₐₓ can be less than about 75%, and in certain embodiments, can be less than about 60%, of the Cₘₐₓ obtained from administering an equivalent dose of the prodrug from an immediate release oral dosage form, and the AUC is substantially the same as the AUC obtained from administering an equivalent dose of the prodrug from an immediate release oral dosage form.

In certain embodiments, the dosage forms of the present disclosure can be administered twice per day, and in certain embodiments, once per day, to provide a therapeutically effective concentration of gabapentin or pregabalin in the systemic circulation of a patient.

### 4.8 Methods of Administration and Doses

The present methods for treatment of vulvodynia comprise administration of a compound of Formula (I) or Formula (II), or a pharmaceutical composition thereof, to a patient in need of such treatment. A compound of Formula (I) or Formula (II), or pharmaceutical compositions thereof can be administered orally, topically, or vaginally. A compound of Formula (I) or Formula (II), or pharmaceutical compositions thereof can also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or pharmaceutical composition thereof. Methods of administration include, but are not limited to, intradermal, intramuscular, intraperitoneal, intravenous, subcutaneous, epidural, oral, sublingual, intranasal, intracerebral, transdermal, rectal, vaginal, by inhalation, or topical, for example, to the vulva, vestibule, or skin.

Dosage forms, upon releasing a GABA analog prodrug of Formulae (I) or (II), can provide the corresponding GABA analog upon administration to a patient. The promoiety or promoieties of the GABA analog prodrug of Formulae (I) or (II) can be cleaved either chemically and/or enzymatically. One or more enzymes present in the stomach, intestinal lumen, intestinal tissue, blood, liver, brain, or any other suitable tissue of a mammal can enzymatically cleave the promoiety or promoieties of the prodrug. If the promoiety or promoieties are cleaved after absorption by the gastrointestinal tract, GABA analog prodrugs of Formulae (I) and (II) can be absorbed into the systemic circulation from the large intestine. In certain embodiments, the promoiety or promoieties are cleaved after absorption by the gastrointestinal tract. In certain embodiments, the promoiety or promoieties are cleaved in the gastrointestinal tract and the corresponding GABA analog is absorbed into the systemic circulation form the large intestine. In certain embodiments, the GABA analog prodrug of Formula (I) or Formula (II) is absorbed into the systemic circulation from the gastrointestinal tract, and the promoiety or promoieties are cleaved in the systemic circulation, after absorption of the GABA analog prodrug from the gastrointestinal tract.

In certain embodiments, the compounds and/or pharmaceutical compositions thereof are delivered via sustained release dosage forms, for example, via oral sustained release dosage forms. In certain embodiments, the compounds and/or pharmaceutical compositions thereof are delivered via topically administered dosage forms. When used to treat vulvodynia a therapeutically effective amount of one or more GABA analog prodrugs of Formulae (I) or (II) can be administered or applied singly or in combination with other agents. A therapeutically effective amount of one or more GABA analog prodrugs of Formulae (I) or (II) can also deliver a compound of the present disclosure in combination with another pharmaceutically active agent, including another compound of the present disclosure. For example, in the treatment of a patient suffering from vulvodynia, a dosage form comprising a GABA analog prodrug of Formulae (I) or (II) can be administered in conjunction with a therapeutic agent known or believed to be capable of treating vulvodynia.

In certain embodiments, a GABA analog prodrug of Formulae (I) or (II) can be provided to a patient by topical administration. For example, a pharmaceutical composition comprising at least one GABA analog prodrug of Formulae (I) or (II) and at least one pharmaceutically acceptable topical vehicle can be formulated in the form of a cream, lotion, ointment, solution, aerosol, spray and the like. It can be desirable that the pharmaceutically acceptable vehicle be selected so as not to exacerbate the symptoms associated with the underlying vulvodynia. For example, certain topical vehicles can cause a burning sensation and/or cause allergic reactions, and are therefore generally avoided for use in topical compositions to be used for treating vulvodynia. The topical formulation can be applied to a surface area of a patient to be treated, for example, by spreading or spraying. The surface area of a patient to be treated can be any area of the vulva exhibiting pain such as the labia, vestibule, vagina, etc. In prophylactic applications, the surface area of a patient to be treated can be, for example, an area of the vulva having a predisposition for exhibiting pain. A compound of Formula (I) or Formula (II) can be used following successful treatment of vulvodynia to delay, in some cases indefinitely, the reoccurrence of vulvar pain symptoms.

The amount of GABA analog prodrug of Formulae (I) or (II) that will be effective in the treatment of vulvodynia in a patient will depend, in part, on the nature of the condition and can be determined by standard clinical techniques known in the art. In addition, in vitro or in vivo assays can optionally be employed to help identify optimal dosage ranges. A therapeutically effective amount of prodrug to be administered can also depend on, among other factors, the subject being treated, the weight of the subject, the severity of the vulvodynia, the manner of administration and the judgment of the prescribing physician.

In some embodiments, the oral sustained release dosage forms are adapted to be administered to a patient 1-3 times per day. In other embodiments, the oral sustained release dosage forms are adapted to be administered to a patient 1-2 times per day. Dosing can be provided alone or in combination with other drugs and may continue as long as required for effective treatment of vulvodynia.

Suitable dosage ranges for oral administration are dependent on the potency of gabapentin or pregabalin (once cleaved from the promoiety) but can range from about 0.1 mg to about 200 mg of drug per kilogram body weight, for example, from about 1 to about 100 mg/kg-body wt. per day. When the GABA analog is gabapentin, examples daily doses of gabapentin in adult patients are about 10 mg/day to about 3600 mg/day and the dose of a compound of Formula (I) may be adjusted to provide an equivalent molar quantity of gabapentin, e.g. examples of daily doses of a compound of Formula (I) in adult patients are about 10 mg-equivalents/day to about 3600 mg-equivalents/day of gabapentin. When the GABA analog is pregabalin, examples of doses for pregabalin in the range of about 10 mg/day to about 1200 mg/day are appropriate, and a dose of a compound of Formula (II) may be adjusted to provide an equivalent molar quantity of pregabalin, e.g., examples of daily doses of a compound of Formula (II) in adult patients are about 10 mg-equivalents/day to about 1200 mg-equivalents/day of pregabalin. Dosage ranges may be readily determined by methods known to the skilled artisan.

In certain embodiments, oral administration of an oral sustained release dosage form comprising a compound of Formulae (I) and/or (II) can provide a therapeutically effected concentration of gabapentin and/or pregabalin, respectively, in the blood or plasma of a patient for a time period of at least about 4 hours after administration of the dosage form, in certain embodiments, for a time period of at least about 8 hours, and in certain embodiments, for a time period of at least about 12 hours.

### 4.9 Combination Therapy

In certain embodiments, a compound of Formula (I) or Formula (II) and/or pharmaceutical compositions thereof can be used in combination therapy with at least one other therapeutic agent that can be a different GABA analog prodrug. The compound of Formula (I) or Formula (II), or pharmaceutical composition thereof and the therapeutic agent can act additively or, in certain embodiments, synergistically. In some embodiments, a compound of Formula (I) or Formula (II), or a pharmaceutical composition thereof can be administered concurrently with the administration of another therapeutic agent. In other embodiments, a compound of Formula (I) or Formula (II) and/or pharmaceutical composition thereof can be administered prior or subsequent to administration of another therapeutic agent.

In certain embodiments, compounds of Formula (I) or Formula (II) and/or pharmaceutical compositions thereof can be administered to a patient for the treatment of vulvodynia in combination with a therapy or treatment known or believed to be effective in the treatment of vulvodynia. Therapies and treatments used to treat vulvodynia include vulvar care measures, topical, oral, and injectable medications, biofeedback, nerve stimulation modulation, physical therapy, pelvic floor therapy, low-oxalate diet and calcium citrate supplementation, surgery, acupuncture, hypnotherapy, nitroglycerin, and botulinum toxin (see, Haefner et al., J. Lower Genital Tract Disease 2005, 9(1), 40-51). Topical medications considered to potentially be effective in treating vulvodynia include ointments and creams containing lidocaine, prilocaine, estrogens, amitrptyiline, or baclofen. Oral medications considered to potentially be effective in treating vulvodynia include, but are not limited to, amitryptyline, nortriptyline, desipramine, venlafaxine, fluoxetine, paroxetine, citalopram, carbamazepine, topiramate, and tramadol.

### 5. EXAMPLES

The invention is further defined by reference to the following examples, which describe preparation of sustained release dosage forms comprising compounds of Formula (I) or Formula (II) and methods of treating vulvodynia comprising administering compounds of Formula (I) or Formula (II). It will be apparent to those skilled in the art that many modifications, both to materials and methods, may be practiced without departing from the scope of the invention.

In the examples below, the following abbreviations have the following meanings. If an abbreviation is not defined, the generally accepted meaning applies.
g=gram
h=hour
kg=kilogram
kV=kilovolt
L=liter
LC/MS=liquid chromatography/mass spectroscopy
mA=milliamps
min=minute
mol=moles
mL=milliliter
mm=millimeter
mmol=millimole
μg=microgram
μL=microliter
μM=micromolar
v/v=volume to volume

### Example 1

### 1-{[(α-Isobutanoyloxyethoxy)carbonyl]aminomethyl}-1-Cyclohexane Acetic Acid via a Trimethylsilyl Ester Intermediate

### Step A

### 1-{[(α-Chloroethoxy)carbonyl]aminomethyl}-1-Cyclohexane Acetic Acid

In a 5-liter, 3-neck, round bottom flask containing dichloromethane (1.6 L) was added gabapentin (120.4 g, 0.704 mol) followed by triethylamine (294 mL, 2.11 mol). Chlorotrimethylsilane (178 mL, 1.40 mol) was slowly added while maintaining the reaction temperature below 15° C. and the resulting suspension was stirred for 30 min. 1-Chloroethyl chloroformate (100 g, 0.704 mol) was then added slowly while maintaining the temperature below 15° C. After the addition was complete, additional triethylamine (88 mL, 0.63 mol) was added and the resulting suspension was stirred at room temperature for 30 min. The resulting silyl ester was converted via acidic work-up to the corresponding acid by washing the reaction mixture with water (2×1 L), followed by 1N HCl (2×2 L) then brine (2×500 mL). After drying over anhydrous sodium sulfate and removal of the solvent in vacuo, the crude product (190 g) was obtained as an orange oil and used in Step B without further purification. ¹H NMR (CDCl₃, 400 MHz): δ 1.41-1.57 (m, 10H), 1.78 (d, 3H), 2.33 (s, 2H), 3.27 (d, 2H), 5.42 (br. S, 1H), 6.55 (q, 1H).

### Step B

### 1-{[(α-Isobutanoyloxyethoxy)carbonyl]aminomethyl}-1-Cyclohexane Acetic Acid (3)

To a 3-liter, 3-neck, round bottom flask was added isobutyric acid (254 g, 2.9 mol) followed by triethylamine (395 mL, 2.84 mol). The reaction mixture was cooled to room temperature and a solution of crude acid from the above reaction step (190 g, 0.69 mol) in dichloromethane (80 mL) was added in a controlled fashion while maintaining the temperature below 30° C. The resulting pale yellow solution was stirred overnight. The reaction mixture was then diluted with one volume of dichloromethane and washed with water (6×500 mL), aqueous potassium bicarbonate (3×500 mL), and brine (2×500 mL). After drying over anhydrous sodium sulfate, removal of the solvent in vacuo afforded the crude product as a dark red oil (87 g). A portion (35 g) of this product was loaded onto an 800 g Biotage™ normal phase silica gel flash column and eluted with 40% diethyl ether in hexane (6 L), which after removal of the solvent in vacuo afforded the product as a colorless oil (13.5 g). This was repeated with a second 35 g portion of crude product yielding a further 13.5 g of 1-{[(α-isobutanoyloxyethoxy)carbonyl]aminomethyl}-1-cyclohexane acetic acid. A sample of the product (25 g) was recrystallized by dissolution in heptane (325 mL) at 70° C., followed by slow cooling to room temperature. The white crystalline product (23 g) was isolated by filtration. Melting point: 63-64° C.

### Example 2

### 1-{[(α-Isobutanoyloxyethoxy)carbonyl]aminomethyl}-1-Cyclohexane Acetic Acid via an Allyl Ester Intermediate

### Step A

### Allyl 1-Aminomethyl-1-Cyclohexane Acetate Hydrochloride

A dry 3 L, three-neck, round-bottomed flash fitted with a magnetic stirring bar and a 500 mL pressure-equalizing addition funnel was flushed with nitrogen gas. The flask was charged with gabapentin (171 g, 1.0 mol) and allyl alcohol (1 L, 852 g, 14.6 mol) and the entire mixture was cooled to 0° C. in an ice-water bath. Thionyl chloride (225 mL, 360 g, 3.0 mol) was added dropwise over a period of 1 h to the stirred solution. The reaction mixture was allowed to stir at room temperature for 16 h, then was diluted with ethyl ether (2 L) and cooled to 0° C. while stirring. After several minutes white crystals formed, which were collected by filtration. The crude product was recrystallized from a 1:3 (v:v) mixture of ethanol and ethyl ether (2 L) to give the product as a white solid (220 g 88%). M.P.: 138-142° C. ¹H NMR (CD₃OD, 400 MHz): δ 1.36-1.54 (m, 10H), 2.57 (s, 2H), 3.05 (s, 2H), 4.61 (d, J=6 Hz, 2H), 5.22 (dd, J=10.4, 1.2 Hz, 1H), 5.33 (dd, J=17.2, 1.4 Hz, 1H), 5.90-6.00 (m, 1H). MS (ESI) m/z 212.0 (M−Cl)⁺.

### Step B

### Allyl 1-{[(α-Chloroethoxy)carbonyl]aminomethyl}-1-Cyclohexane Acetate

To a solution of the above hydrochloride salt (220 g, 0.89 mol) in dichloromethane (1 L) was slowly added 1-chloroethyl chloroformate (101.7 mL, 132.3 g, 0.92 mol). The reaction mixture was cooled to 0° C. and 4-methylmorpholine (205 mL, 188.9 g, 1.87 mol) was slowly added over a period of 1 h while maintaining a temperature of less than 10° C. The resulting turbid solution was stirred at room temperature for 1 h. Ethanol (150 mL) was added and the reaction mixture was stirred at room temperature for 1 h. The reaction mixture was then diluted with ether (2.5 L), washed with water (1 L) and brine (1 L). The organic phase was dried over sodium sulfate and concentrated to give the title compound as a light yellow viscous liquid (282 g, 100%). ¹H NMR (CDCl₃, 400 MHz): δ 1.35-1.58 (m, 10H), 1.78 (d, J=5.6 Hz, 3H), 2.32 (s, 2H), 3.22 (d, J=6.8 Hz, 2H), 4.57 (d, J=5.6 Hz, 2H), 5.25 (dd, J=10.4, 1 Hz, 1H), 5.32 (dd, J=17.2, 1.6 Hz, 1H), 5.52 (br, 1H, NH), 5.90-5.94 (m, 1H), 6.54 (q, J=5.6 Hz, 1H).

### Step C

### Allyl 1-{[(α-Isobutanoyloxyethoxy)carbonyl]aminomethyl}-1-Cyclohexane Acetate

To a mixture of isobutyric acid (432 mL, 391.5 g, 4.4 mol) and 4-methylmorpholine (488 mL, 449 g, 4.4 mol) was added a solution of the chlorocarbamate from the previous step (282 g, 0.88 mol) in isobutyric acid (432 mL, 391.5 g, 4.4 mol). The addition occurred at 0° C. over a period of 30 min. The resulting turbid solution was stirred at room temperature for 16 h. The reaction mixture was diluted with ether (2.5 L) and washed with water (3×500 mL) followed by 10% aqueous potassium bicarbonate (6×500 mL) then brine (500 mL). The organic phase was dried over sodium sulfate and concentrated to yield the title compound as a viscous liquid (328 g, 100%). ¹H NMR (CDCl₃, 400 MHz): δ 1.15 (d, J=7.2 Hz, 6H), 1.35-1.58 (m, 10H), 2.31 (s, 2H), 2.51 (m, 1H), 3.19 (d, J=5.6 Hz, 2H), 4.56 (d, J=5.6 Hz, 2H), 5.24 (dd, J=10, 1 Hz, 1H), 5.32 (dd, J=17, 1.2 Hz, 1H), 5.35 (br, 1H), 5.84-5.94 (m, 1H), 6.78 (q, J=5.6 Hz, 1H). MS (ESI) m/z 392.24 (M+H)⁺.

### Step D

### Deprotection of Allyl 1-{[(α-Isobutanoyloxyethoxy)carbonyl]-aminomethyl}-1-Cyclohexane Acetate

To a stirred suspension of ammonium formate (112 g, 1.7 mol) in ethanol (500 mL) was added the above allyl ester (328 g, 0.88 mol) together with 10% Pd/C (15 g) under a nitrogen atmosphere. After 6 h, the reaction mixture was worked-up by filtering off the catalyst. The catalyst was washed with ethanol (2×250 mL) and the filtrates were combined and evaporated. The crude product was dissolved in ether (2 L) and the organic phase was washed with 2N HCl (2×2 L) to convert the ammonium salt into the acid form, followed by washing with water (1 L) and brine (1 L). The ether layer was dried over sodium sulfate and concentrated to give 1-{[(α-isobutanoyloxyethoxy)carbonyl]-aminomethyl}-1-cyclohexane acetic acid as a viscous liquid (240 g, 82%).

### Step E:

### Crystallization of 1-{[(α-Isobutanoyloxyethoxy)carbonyl]-aminomethyl}-1-Cyclohexane Acetic Acid

A 3 L round-bottom flask was equipped with a heating oil bath, a nitrogen inlet adapter, an internal thermometer, an overhead mechanical stirrer, and a reflux condenser. The flask was flushed with nitrogen and charged with a 1:10 (v:v) mixture of ethyl acetate:heptane (1.2 L) and the crude product from the preceding reaction (240 g). The flask was heated until the product dissolved, then cooled according to the following schedule:

| Time | Temp (° C.) | | |
|---|---|---|---|
| (min) | (Internal) | Appearance | Remarks |
| 0 | 18 | Solid in solvent | Started heating oil bath |
| 10 | 48 | Turbid | Slow dissolution of product |
| 20 | 58 | Clear solution | Turn off oil bath |
| 25 | 60 | Clear solution | Maximum temp. reached |
| 45 | 43 | Turbid | Compound crystallizing |
| 60 | 36 | Milky solution | Seeded with pure ref. material |
| 90 | 24 | Solid in solution | |

The flask was then cooled to 4° C. overnight with stirring (cooling improves the yield) The product was filtered and washed with heptane (2×100 mL), then dried under e (25 mm of Hg (0.033 atm)) at 30° C. for 18 h to yield 1-{[(α-isobutanoyloxyethoxy)carbonyl]aminomethyl}-1-cyclohexane acetic acid (185 g) as a white crystalline solid.

### Example 3

### X-Ray Powder Diffraction Analysis of Crystalline 1-{[(α-Isobutanoyloxyethoxy)carbonyl]aminomethyl}-1-Cyclohexane Acetic Acid

X-ray powder diffractograms (XRPDs) of crystalline samples of 1-{[(α-isobutanoyloxyethoxy)carbonyl]aminomethyl}-1-cyclohexane acetic acid produced according to Examples 1 and 2 above were measured with a Bruker D8 Discover X-ray powder diffractometer using Cu Kα radiation. The instrument is equipped with parallel beam optics and a two-dimensional HI-STAR area detector. The tube voltage and amperage were set to 40 kV and a 40 mA repectively. The collimated X-ray beam was reduced to a spot size of about 0.5 mm in diameter. The area detector was placed 15 cm from the center of the goniometer and the angular resolution is approximately 0.033°/pixel. The detector covered a range of 35° in 2-theta (2θ) within one frame. The angle between the X-ray beam and the horizontal sample plate was set to 4° and the center of the area detector was set to an angle of 18°. This geometry allowed the measurement of 2-theta from 4.5° to 39.5° within one frame. Typical averaging time was 3 minutes for each XRPD pattern collected. A corundum sample (NIST 1976) was used to calibrate the XRPD instrument. Both samples gave equivalent diffractogram patterns.

### Example 4

### Melting Point and Differential Scanning Calorimetry Analysis of Crystalline 1-{[(α-Isobutanoyloxyethoxy)carbonyl]aminomethyl}-1-Cyclohexane Acetic Acid

Melting points of crystalline samples of 1-{[(α-isobutanoyloxyethoxy)carbonyl]aminomethyl}-1-cyclohexane acetic acid produced according to Examples 1 and 2 above were measured using an Electrothermal 9200 melting point apparatus and determined to be 63-64° C.

Differential scanning calorimetry (DSC) analysis of crystalline samples of 1-{[(α-isobutanoyloxyethoxy)carbonyl]aminomethyl}-1-cyclohexane acetic acid produced according to Examples 1 and 2 above were measured using a Perkin Elmer Series 7 instrument, scanning from 25° C. to 250° C. at a scan rate of 5° C./minute. A test portion of the sample was placed in an aluminum pan and the cap crimped to eliminate any visible seam between the cap and the pan. An empty pan was prepared in the same manner as a blank. The pans were placed in the Differential Scanning Calorimeter. The material was run at the appropriate temperature program (Equilibration at Initial Temp, Isothermal, Ramp Rate, Final Temp). DSC analysis showed an endothermic transition with an onset temperature of 58.3° C. and a ΔH of 72.39 J/g. At the peak endotherm of 63-64° C. the sample visibly melted.

### Example 5

### {[(1-Isobutanoyloxyethoxy)carbonyl]aminomethyl}-1-Cyclohexane Acetic Acid

To a solution of gabapentin (6.8 g, 0.04 mol) in water (40 mL) was added a solution of [(1-isobutanoyloxyethoxy)carbonyloxy]succinimide (10 g, 0.036 mol) in acetonitrile (40 mL) over a period of 30 minutes. The reaction was stirred at ambient temperature for 3 hours. The reaction mixture was diluted with methyl tert-butyl ether (200 mL), washed with water (2×100 mL) and brine (50 mL). The organic phase was separated, dried over anhydrous sodium sulfate, filtered, and concentrated in vacuo to afford the title compound as a white solid (12 g, quantitative).

The following procedure was used to crystallize the title compound. The solid compound (12 g) was suspended in methylcyclohexane: methyl tert-butyl ether 10:1 (60 mL). The suspension was slowly heated up to 50° C. over a period of 30 min. The clear solution was then allowed to cool to room temperature. The turbid mixture was seeded with 5 mg of the title compound in crystalline form. The mixture was further cooled to 0-4° C. for 2 h. The solid product was filtered and washed with methylcyclohexane (2×10 mL) to yield the title compound as a white crystalline solid (10 g, 83% yield). The crystalline solid material had a melting point of about 64-66° C. as measured by open capillary melting point determination.

### Example 6

### Preparation of a Sustained Release Oral Dosage Form of 1-{[(α-Isobutanoyloxyethoxy)carbonyl]aminomethyl}-1-Cyclohexane Acetic Acid (3)

Sustained release oral dosage forms containing the gabapentin prodrug, 1-{[(α-isobutanoyloxyethoxy)carbonyl]aminomethyl}-1-cyclohexane acetic acid (compound (3)) , was prepared according the procedure disclosed in Cundy, U.S. Application Publication No. 2006/0141034. Oral sustained release dosage form tablets containing compound (3) were made having the ingredients shown in Table I:

TABLE I

| | | Amount/Tablet | % Composition | Ingredient |
|---|---|---|---|---|
| Ingredient | Manufacturer | (mg/tablet) | (w/w) | Category |
| Compound (3) | XenoPort | 600.00 | 45.80 | Prodrug |
| | (Santa Clara, | | | |
| | CA) | | | |
| Dibasic Calcium | Rhodia | 518.26 | 39.56 | Diluent |
| Phosphate, USP | (Chicago, IL) | | | |
| Glyceryl | Gattefosse | 60.05 | 4.58 | Lubricant/ |
| Behenate, NF | (Saint Pirest, | | | Release |
| | Cedex, France) | | | controlling |
| Talc, USP | Barrett Minerals | 80.02 | 6.11 | Anti-adherent |
| | (Mount Vernon, | | | |
| | IN) | | | |
| Colloidal Silicon | Cabot (Tuscola, | 5.43 | 0.41 | Glidant |
| Dioxide, NF | IL) | | | |
| Sodium Lauryl | Fisher | 24.00 | 1.84 | Surfactant |
| Sulfate, NF | (Fairlawn, NJ) | | | |
| Magnesium | Mallinckrodt | 22.22 | 1.69 | Lubricant |
| Stearate, NF | (Phillipsburg, | | | |
| | NJ) | | | |
| Total Weight | | 1310 | 100 | |
| (mg) | | | | |

The tablets were made according to the following steps. Compound (3), dibasic calcium phosphate, glyceryl behenate, talc, and colloidal silicon dioxide were weighed out, screened through a #20 mesh screen and mixed in a V-blender for 15 minutes. The slugging portion of the sodium lauryl sulfate was weighed and passed through a #30 mesh screen. The slugging portion of the magnesium stearate was weighed and passed through a #40 mesh screen. Screened sodium lauryl sulfate and magnesium stearate were added to the V-blender and blended for 5 minutes. The blend was discharged and compressed into slugs of approximately 400 mg weight on a tablet compression machine. The slugs were then passed through a Comil 194 Ultra mill (Quadro Engineering, Inc., Millburn, N.J.) to obtain the milled material for further compression. The tableting portion of the sodium lauryl sulfate was weighed and passed through a #30 mesh screen. The tableting portion of the magnesium stearate was weighed and passed through a #40 mesh screen. The milled material and the tableting portions of the sodium lauryl sulfate and magnesium stearate were added to the V-blender and blended for 3 minutes. The blended material was discharged and compressed to form tablets having a total weight of 1310 mg and a compound (3) loading of 600 mg (45.8 wt %). The tablets had a mean final hardness of 16.1 to 22.2 kp (158 to 218 Newtons).

### Example 7

### Pharmacokinetics of Orally Administered 1-{[(α-Isobutanoyloxyethoxy)carbonyl]aminomethyl}-1-Cyclohexane Acetic Acid (3)

A randomized, crossover, fed/fasted single-dose study of the safety, tolerability, and pharmacokinetics of oral administration of 1-{[(α-isobutanoyloxyethoxy)carbonyl]aminomethyl}-1-cyclohexane acetic acid (3) in healthy adult subjects was conducted. The oral sustained release dosage form of Example 6 was used in this study. The study was designed to evaluate the performance of this formulation in humans in comparison with the commercial gabapentin capsule formulation (Neurontin®, Pfizer). Twelve healthy adult volunteers (7 males and 5 females) participated in the study. Mean body weight was 75.6 kg. All subjects received two different treatments in a random order with a one-week washout between treatments. The two treatments were: (A) a single oral dose of Example 1 tablets (2×600 mg) after an overnight fast; and (B) a single oral dose of Example 1 tablets (2×600 mg) after a high fat breakfast.

Blood and plasma samples were collected from all subjects prior to dosing, and at 0.5, 1, 1.5, 2, 3, 4, 6, 8, 12, 18, 24, and 36 hours after dosing. Urine samples were collected from all subjects prior to dosing, and complete urine output was obtained at the 0-4 h, 4-8 h, 8-12 h, 12-18 h, 18-24 h, and 24-36 h intervals after dosing. Blood samples were quenched immediately with methanol and stored frozen at ≦70° C. Sample aliquots were prepared for analysis of gabapentin and compound (3) using sensitive and specific LC/MS/MS methods.

The mean±SD Cₘₐₓ for gabapentin in blood after oral dosing of the tablets (fasted) was 4.21±1.15 μg/mL. Following administration of the tablets after a high fat breakfast, the Cₘₐₓ of gabapentin in blood was further increased to 6.24±1.55 μg/mL. The mean±SD AUC for gabapentin in blood after oral dosing of the tablets (fasted) was 54.5±12.2 μg·h/mL. Following administration of the tablets after a high fat breakfast, the AUC of gabapentin in blood was further increased to 83.0±21.8 μg·h/mL. In the presence of food, exposure to gabapentin after oral administration of the tablets increased an additional 52% compared to that in fasted subjects.

The time to peak blood levels (T^{max}) of gabapentin was significantly delayed after oral administration of the tablets. In fasted subjects, oral administration of the tablets gave a gabapentin Tₘₐₓ of 5.08±1.62 h. This compares to a typical Tₘₐₓ of immediate release gabapentin of about 2-4 h. The gabapentin Tₘₐₓ after oral administration of the tablets was further delayed to 8.40±2.07 h in the presence of food. The apparent terminal elimination half-life for gabapentin in blood was similar for all treatments: 6.47±0.77 h for the tablets in fasted subjects, and 5.38±0.80 h for the tablets in fed subjects.

Following oral administration of the tablets, the percent of the gabapentin dose recovered in urine was 46.5±15.8% for fasted subjects and 73.7±7.2% for fed subjects.

Exposure to intact prodrug in blood after oral administration of the tablets was low. After oral dosing of the tablets in fasted subjects, concentrations of intact compound (3) in blood reached a maximum of 0.040 μg/mL, approximately 1.0% of the corresponding peak gabapentin concentration. Similarly, the AUC of compound (3) in blood of these subjects was 0.3% of the corresponding AUC of gabapentin in blood. After oral dosing of the tablets in fed subjects, concentrations of intact compound (3) in blood reached a maximum of 0.018 μg/mL, approximately 0.3% of the corresponding peak gabapentin concentration. Similarly, the AUC of compound (3) in blood of these subjects was <0.1% of the corresponding AUC of gabapentin in blood.

### Example 8

### Administration of 1-{[(α-Isobutanoyloxyethoxy)carbonyl]-aminomethyl}-1-Cyclohexane Acetic Acid (3) for the Treatment of Vulvodynia

A placebo-controlled, cross-over clinical trial is conducted to assess the effects of the prodrug 1-{[(α-isobutanoyloxyethoxy)carbonyl]-aminomethyl}-1-cyclohexane acetic acid on sensory pain symptoms in patients with vulvodynia. The clinical trial can be similar to the general method described by Friedrich, J. Reprod. Med. 1987, 32, 110-14, for assessing vulvar vestibulitis, or by M. C. Rowbotham and H. L. Fields, Pain 1989, 38, 297-301, for assessing neuropathic pain. Briefly, sixty patients with either vulvar vestibulitis or dysesthetic vulvodynia are randomized and treated for 3 months with either a sustained release oral dosage comprising the prodrug as disclosed in Example 6, a topical dosage form comprising the prodrug, or a placebo. After a 1-week washout, the patients are crossed over to alternative treatment for 6 weeks. Patients are rated at baseline and at scheduled intervals during the study.

For oral administration, after a two week baseline screening assessment, the prodrug 1-{[(α-isobutanoyloxyethoxy)carbonyl]aminomethyl}-1-cyclohexane acetic acid, formulated as a sustained release dosage form according to Example 6 containing 600 mg drug, is administered two capsules twice daily (2400 mg/day, equal to ˜1200 mg gabapentin equivalents/day) for three months.

Prior to topical administration, the painful area to be treated is noted and photographed based on the subject's report of (1) the borders of the area of sensory abnormality, and (2) the area of greatest pain. Ointment comprising the prodrug is then applied once or twice a day to the affected area in the amount of 1-2 g of ointment per 10 cm² of skin. The subjects make ratings of pain, pain relief, and side effects at intervals after initial application of the ointment.

Pain intensity is assessed using a horizontal 100 mm visual analog scale (VAS). The subject indicates the severity of his or her pain with a mark along the line between “no pain” (0 mm) and “worst pain imaginable” (100 mm). Prior to application, VAS scores are obtained 3 times over a 45-minute period; once before quantitative sensory testing (QST) and two times following QST. After application, VAS scores are obtained at 30 minutes, 1 hour, 2 hours, 4 hours, 6 hours, 9 hours, and 12 hours.

Pain relief is assessed using a category scale consisting of 6 sentences indicating that: the pain is increasing (score 0), “no” pain relief (1), “slight” pain relief (2), “moderate” pain relief (3), “a lot” of pain relief (4), and “complete” relief of pain (5). As the scale is designed to assess changes only, there is no baseline pre-application rating. After topical application, category relief scores are obtained at 30 minutes, 1 hour, 2 hours, 4 hours, 6 hours, 9 hours, and 12 hours.

A statistical analysis of the data obtained is conducted using the method of analysis of variance whenever possible. This is accomplished using the Statistical Analysis System (SAS) v. 6.04, under the procedure General Linear Models. An overall F-test is conducted to determine if there are differences among the three treatments. Additionally, pairwise contrast tests between treatments are performed to evaluate the statistical significance between pairs of treatments. The difference between two treatments (F-test) is considered statistically significant if both the overall and pairwise p-values are less than or equal to 0.05. For pain intensity VAS scores and QST data, the pairwise comparisons are made at individual time points in addition to the overall F-test.

A positive result for the prodrug is associated with reduced symptoms on all rating scales when compared with the placebo. Similar methods can be used to evaluate the therapeutic efficacy of other compounds of Formula (I) or Formula (II) for treating vulvodynia.

Finally, it should be noted that there are alternative ways of implementing the present invention. Accordingly, the present embodiments are to be considered as illustrative and not restrictive, and the invention is not to be limited to the details given herein, but may be modified within the scope and equivalents of the claims issuing here from.

What is claimed is:

1. A method of treating vulvodynia in a patient, comprising administering to a patient in need of such treatment a therapeutically effective amount of a compound chosen from Formula (I), Formula (II): 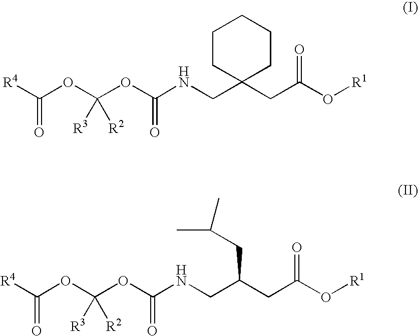
a pharmaceutically acceptable salt of any of the foregoing, a pharmaceutically acceptable solvate of any of the foregoing, and a pharmaceutically acceptable N-oxide of any of the foregoing, wherein:
R¹ is chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl;
R² and R³ are independently chosen from hydrogen, alkyl, substituted alkyl, alkoxycarbonyl, substituted alkoxycarbonyl, aryl, substituted aryl, arylalkyl, substituted arylalkyl, carbamoyl, substituted carbamoyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl, or R² and R³ together with the carbon atom to which they are bonded form a cycloalkyl, substituted cycloalkyl, cycloheteroalkyl, or substituted cycloheteroalkyl ring; and
R⁴ is chosen from acyl, substituted acyl, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

2. The method of claim 1, wherein R¹ is hydrogen.

3. The method of claim 1, wherein R² and R³ are independently chosen from hydrogen and C₁₋₆ alkyl.

4. The method of claim 1, wherein R³ is chosen from methyl, ethyl, n-propyl, isopropyl, n-butyl, isobutyl, and sec-butyl, and R² is hydrogen.

5. The method of claim 1, wherein R⁴ is chosen from C₁₋₆ alkyl and C₁₋₆ substituted alkyl.

6. The method of claim 1, wherein R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

7. The method of claim 1, wherein R¹ and R² are each hydrogen, R³ is C₁₋₆ alkyl, and R⁴ is chosen from C₁₋₆ alkyl and C₁₋₆ substituted alkyl.

8. The method of claim 1, wherein R¹ and R² are each hydrogen, R³ is chosen from methyl, ethyl, n-propyl, isopropyl, n-butyl, isobutyl, and sec-butyl, and R⁴ is chosen from methyl, ethyl, n-propyl, isopropyl, n-butyl, isobutyl, sec-butyl, n-pentyl, isopentyl, sec-pentyl, neopentyl, and 1,1-diethoxyethyl.

9. The method of claim 1, wherein each substituent is independently chosen from halogen, —NH₂, —OH, —CN, —COOH, —C(O)NH₂, —C(O)OR⁵, and —NR⁵₃⁺, and each R⁵ is independently C₁₋₃ alkyl.

10. The method of claim 5, wherein each substituent is independently chosen from halogen, —NH₂, —OH, —CN, COOH, —C(O)NH₂, —C(O)OR⁵, and —NR⁵₃⁺, and each R⁵ is independently C₁₋₃ alkyl.

11. The method of claim 7, wherein each substituent is independently chosen from halogen, —NH₂, —OH, —CN, —COOH, —C(O)NH₂, —C(O)OR⁵, and —NR⁵₃⁺, and each R⁵ is independently C₁₋₃ alkyl.

12. The method of claim 1, wherein the compound is a compound of Formula (I) and is chosen from:
1-{[(α-acetoxyethoxy)carbonyl]aminomethyl}-1-cyclohexane acetic acid;
1-{[(α-propanoyloxyethoxy)carbonyl]aminomethyl}-1-cyclohexane acetic acid;
1-{[(α-butanoyloxyethoxy)carbonyl]aminomethyl}-1-cyclohexane acetic acid;
1-{[(α-isobutanoyloxyethoxy)carbonyl]aminomethyl}-1-cyclohexane acetic acid;
1-{[(α-pivaloxyethoxy)carbonyl]aminomethyl}-1-cyclohexane acetic acid;
1-{[(α-acetoxymethoxy)carbonyl]aminomethyl}-1-cyclohexane acetic acid;
1-{[(α-propanoyloxymethoxy)carbonyl]aminomethyl}-1-cyclohexane acetic acid;
1-{[(α-butanoyloxymethoxy)carbonyl]aminomethyl}-1-cyclohexane acetic acid;
1-{[(α-isobutanoyloxymethoxy)carbonyl]aminomethyl}-1-cyclohexane acetic acid;
1-{[(α-pivaloxymethoxy)carbonyl]aminomethyl}-1-cyclohexane acetic acid;
1-{[(α-acetoxypropoxy)carbonyl]aminomethyl}-1-cyclohexane acetic acid;
1-{[(α-propanoyloxypropoxy)carbonyl]aminomethyl}-1-cyclohexane acetic acid;
1-{[(α-butanoyloxypropoxy)carbonyl]aminomethyl}-1-cyclohexane acetic acid;
1-{[(α-isobutanoyloxypropoxy)carbonyl]aminomethyl}-1-cyclohexane acetic acid;
1-{[(α-pivaloxypropoxy)carbonyl]aminomethyl}-1-cyclohexane acetic acid;
1-{[(α-acetoxyisopropoxy)carbonyl]aminomethyl}-1-cyclohexane acetic acid;
1-{[(α-propanoyloxyisopropoxy)carbonyl]aminomethyl}-1-cyclohexane acetic acid;
1-{[(α-butanoyloxyisopropoxy)carbonyl]aminomethyl}-1-cyclohexane acetic acid;
1-{[(α-isobutanoyloxyisopropoxy)carbonyl]aminomethyl}-1-cyclohexane acetic acid;
1-{[(α-pivaloxyisopropoxy)carbonyl]aminomethyl}-1-cyclohexane acetic acid;
1-{[(α-acetoxybutoxy)carbonyl]aminomethyl}-1-cyclohexane acetic acid;
1-{[(α-propanoyloxybutoxy)carbonyl]aminomethyl}-1-cyclohexane acetic acid;
1-{[(α-butanoyloxybutoxy)carbonyl]aminomethyl}-1-cyclohexane acetic acid;
1-{[(α-isobutanoyloxybutoxy)carbonyl]aminomethyl}-1-cyclohexane acetic acid;
1-{[(α-pivaloxybutoxy)carbonyl]aminomethyl}-1-cyclohexane acetic acid;
a pharmaceutically acceptable salt of any of the foregoing, a pharmaceutically acceptable solvate of any of the foregoing, and a pharmaceutically acceptable N-oxide of any of the foregoing.

13. The method of claim 1, wherein the compound is a compound of Formula (I) and is 1-{[(α-isobutanoyloxyethoxy)carbonyl]aminomethyl}-1-cyclohexane acetic acid, a pharmaceutically acceptable salt thereof, a pharmaceutically acceptable solvate of any of the foregoing, or a pharmaceutically acceptable N-oxide of any of the foregoing.

14. The method of claim 13, wherein the compound of Formula (I) {[(α-isobutanoyloxyethoxy)carbonyl]aminomethyl}-1-cyclohexane acetic acid is crystalline.

15. The method of claim 1, wherein the compound is a compound of Formula (II) and is chosen from:
3-{[(α-acetoxyethoxy)carbonyl]aminomethyl}-5-methyl hexanoic acid;
3-{[(α-propanoyloxyethoxy)carbonyl]aminomethyl}-5-methyl hexanoic acid;
3-{[(α-butanoyloxyethoxy)carbonyl]aminomethyl}-5-methyl hexanoic acid;
3-{[(α-isobutanoyloxyethoxy)carbonyl]aminomethyl}-5-methyl hexanoic acid;
3-{[(α-pivaloxyethoxy)carbonyl]aminomethyl}-5-methyl hexanoic acid;
3-{[(α-acetoxymethoxy)carbonyl]aminomethyl}-5-methyl hexanoic acid;
3-{[(α-propanoyloxymethoxy)carbonyl]aminomethyl}-5-methyl hexanoic acid;
3-{[(α-butanoyloxymethoxy)carbonyl]aminomethyl}-5-methyl hexanoic acid;
3-{[(α-isobutanoyloxymethoxy)carbonyl]aminomethyl}-5-methyl hexanoic acid;
3-{[(α-pivaloxymethoxy)carbonyl]aminomethyl}-5-methyl hexanoic acid;
3-{[(α-acetoxypropoxy)carbonyl]aminomethyl}-5-methyl hexanoic acid;
3-{[(α-propanoyloxypropoxy)carbonyl]aminomethyl}-5-methyl hexanoic acid;
3-{[(α-butanoyloxypropoxy)carbonyl]aminomethyl}-5-methyl hexanoic acid;
3-{[(α-isobutanoyloxypropoxy)carbonyl]aminomethyl}-5-methyl hexanoic acid;
3-{[(α-pivaloxypropoxy)carbonyl]aminomethyl}-5-methyl hexanoic acid;
3-{[(α-acetoxyisopropoxy)carbonyl]aminomethyl}-5-methyl hexanoic acid;
3-{[(α-propanoyloxyisopropoxy)carbonyl]aminomethyl}-5-methyl hexanoic acid;
3-{[(α-butanoyloxyisopropoxy)carbonyl]aminomethyl}-5-methyl hexanoic acid;
3-{[(α-isobutanoyloxyisopropoxy)carbonyl]aminomethyl}-5-methyl hexanoic acid;
3-{[(α-pivaloxyisopropoxy)carbonyl]aminomethyl}-5-methyl hexanoic acid;
3-{[(α-acetoxybutoxy)carbonyl]aminomethyl}-5-methyl hexanoic acid;
3-{[(α-propanoyloxybutoxy)carbonyl]aminomethyl}-5-methyl hexanoic acid;
3-{[(α-butanoyloxybutoxy)carbonyl]aminomethyl}-5-methyl hexanoic acid;
3-{[(α-isobutanoyloxybutoxy)carbonyl]aminomethyl}-5-methyl hexanoic acid;
3-{[(α-pivaloxybutoxy)carbonyl]aminomethyl}-5-methyl hexanoic acid;
a pharmaceutically acceptable salt of any of the foregoing, a pharmaceutically acceptable solvate of any of the foregoing, and a pharmaceutically acceptable N-oxide of any of the foregoing.

16. The method of claim 1, wherein the compound is a compound of Formula (II) and is 3-{[(α-isobutanoyloxyethoxy)carbonyl]aminomethyl}-5-methyl hexanoic acid, a pharmaceutically acceptable salt thereof, a pharmaceutically acceptable solvate of any of the foregoing, or a pharmaceutically acceptable N-oxide of any of the foregoing.

17. The method of claim 1, wherein the compound is administered in an amount ranging from about 10 mg equivalents to about 3600 mg equivalents of gabapentin or pregabalin per day.

18. The method of claim 1, wherein the compound is administered orally.

19. The method of claim 17, wherein the compound is administered in a sustained release oral dosage form.

20. The method of claim 19, wherein a therapeutically effective amount of gabapentin or pregabalin is maintained in the blood or plasma of the patient for a period of at least about 4 hours after administrating the compound.

21. The method of claim 20, wherein the therapeutically effective amount of gabapentin or pregabalin is maintained in the blood or plasma of the patient for a period of at least about 8 hours after administrating the compound.

22. The method of claim 20, wherein the therapeutically effective amount of gabapentin or pregabalin is maintained in the blood or plasma of the patient for a period of at least 12 hours after administrating the compound.

23. The method of claim 1, wherein the compound is administered topically.

24. The method of claim 1, wherein the compound is formulated with a pharmaceutically acceptable vehicle.
